(19) United States
(12) Patent Application Publication
(10) Pub. No.: US 2010/0216130 A1
(43) Pub. Date: Aug. 26, 2010

(54) SINGLE NUCLEOTIDE POLYMORPHISMS ASSOCIATED WITH AMYOTROPHIC LATERAL SCLEROSIS

(75) Inventors: Dietrich Stephan, Foster City, CA (US); Matthew Huentelman, Phoenix, AZ (US); Travis Dunckley, Phoenix, AZ (US); Stephen Gately, Scottsdale, AZ (US)

Correspondence Address: JEFFREY M. JACKSON, 445 N. 5th Street, Phoenix, AZ, 85004, US

(73) Assignee: Translational Genomics Research Institute

(21) Appl. No.: 12/516,100

(22) PCT Filed: Nov. 29, 2007
(86) PCT No.: PCT/US2007/085926

§ 371 Date: May 22, 2009

Related U.S. Application Data
(60) Provisional application No. 60/868,085, filed on Nov. 30, 2006.

(51) Int. Cl. C12Q 1/68

(52) U.S. Cl. 435/6

(57) ABSTRACT

Methods for determining the genetic predisposition of a human subject to developing ALS are provided herein. These methods include methods for determining the genetic predisposition to any form of ALS, as well as specific methods for determining the genetic predisposition to early onset, late onset, bulbar onset and limb onset ALS. The method can detect amyotrophic lateral sclerosis in a human subject or a specific form of ALS in the subject (early onset, late onset, bulbar onset or limb onset). The method can also detect the risk of developing amyotrophic lateral sclerosis (ALS) in a human subject. The methods utilize the detection of one or more haplotype bocks comprising tag single nucleotide polymorphisms (SNPs). In several embodiments, the methods including detecting the presence of one or more tag SNPs.

### PRIORITY CLAIM

This application claims the benefit of U.S. Provisional Application No. 60/868,085, filed Nov. 30, 2006, which is incorporated herein by reference.

### FIELD

This disclosure relates to the field of individualized medicine, specifically to the detection of Amyotrophic Lateral Sclerosis (ALS) and the determination of the prognosis of a subject with ALS.

### BACKGROUND

Amyotrophic Lateral Sclerosis is the most common motor neuron disease with an incidence of 1-2 per 100,000 and a lifetime risk of 1:800. ALS is characterized by a progressive loss of motor neurons from the spinal cord, brainstem, and cerebral cortex, eventually leading to paralysis and death within two to five years of diagnosis. Approximately ten percent of ALS cases are familial forms resulting from highly penetrant monogenic disease-causing mutations. Some specific genes associated with these forms of ALS have been identified, including many known mutations in the superoxide dismutase 1 gene, SOD1 (Pasinelli and Brown, Nat Rev Neurosci. 2006; 7:710-23). In addition to SOD1, there are two recessive loci, ALS2 (alsin) and ALS5, as well as five additional dominant loci ALS3, ALS4 (SETX), ALS6, ALS7, ALS8 (VAPB) that have been implicated in familial ALS. Four additional dominant loci have been linked to variant forms of motor neuron disease, including two ALS-fronto-temporal dementia loci on chromosome 9q21-22 and 9p21.3-13.3, ALS with dementia, Parkinsonism (MAPT on chromosome 17q21), and DCTN1 on chromosome 2p13 linked to progressive lower motor neuron disease (Pasinelli and Brown, supra).

Little is known about the specific genes that contribute to the development of spontaneous (s)ALS. Moreover, despite extensive study of familial ALS mutations in vitro and in animal models of ALS, it remains unclear what the key events are in the initiation and progression of sporadic ALS disease. Pathologically, ALS is characterized by loss of motor neurons from the motor cortex, brainstem and spinal ventral horns. Ubiquitinated inclusions can be found histopathologically in lower motor neurons, although their role in disease initiation and progression is unclear (Ince, Neuropathology. In: Brown R H Jr, Meininger K, Swash M (eds) Amyotrophic lateral sclerosis. Martin Dunitz, London, 2000; pp 83-112). Numerous mechanisms have been implicated in the selective motor neuron degeneration in sALS, including oxidative damage, excitotoxicity, apoptosis, cytoskeletal function, axonal transport defects, inflammation, protein processing and degradation defects, and mitochondrial dysfunction (Cleveland and Rothstein, Nature Rev Neurosci 2001; 2:806-19; Bruijn et al., Ann Rev Neurosci 2004; 27:723-49). Identifying the specific genetic variants associated with sALS improves the understanding of the fundamental disease mechanisms.

There is a need to identify genes that are statistically significantly associated with ALS, in order to detect a genetic predisposition to ALS. There is a need to identify these genes in order to identify new targets for the treatment of ALS, and to allow for the development of individualized therapeutic protocols. In addition, there is a need to detect ALS early in subjects, and to identify those subjects that will develop specific forms of ALS, such as bulbar onset, limb onset, early onset and late onset, so that these individuals obtain early intervention.

### SUMMARY

Methods are provided herein for determining the genetic predisposition of a human subject to developing ALS. In several embodiments, the method determines the genetic predisposition of a subject to any type of ALS. In additional embodiments, the method determines the genetic predisposition to a specific form of ALS in the subject (early onset, late onset, bulbar onset or limb onset). The methods disclosed herein can detect ALS, or can detect the risk of developing ALS in a human subject. In some embodiments, the methods utilize the detection of one or more haplotype bocks comprising a tag single nucleotide polymorphism (SNP). In additional embodiments, the method includes detecting the presence of one or more tag SNPs. The methods can include detecting multiple haplotype blocks and/or multiple tag SNPs.

The foregoing and other features and advantages will become more apparent from the following detailed description of several embodiments, which proceeds with reference to the accompanying figures.

### BRIEF DESCRIPTION OF THE FIGURES

FIG. 1 is a graph showing the fine-mapping of the FLJ10986 locus in the ALS validation population. Individual genotyping of an independent ALS population of 766 individuals confirmed pooling based screening results and identified rs6700125 and rs6690993 as associated with sporadic ALS. Subsequently, 71 additional flanking SNPs were selected to fine map the associated region. Results showed significant association with ALS for four additional SNPs. One SNP lies in the promoter region of FLJ10986, two lie within intron 1, and 1 lies within intron 2 of the FLJ10986 gene. The continuous red horizontal line indicates the p=0.005 level of significance. Chromosome position in base pairs is indicated on the x axis. Intronic regions of FLJ10986 are indicated in gray and short vertical lines indicate exons. The FLJ10986 gene continues nearly 250 kb beyond the region shown. The lower panel visualizes the haplotype structure of the examined region as assessed by Haploview 3.32. Red squares indicate regions of high LD (D′>0.8).

FIGS. 2A-2C are digital images and bar graphs representing FLJ10986 immunoblot analysis. FIG. 2A is a digital image showing FLJ10986 is expressed in multiple human tissues, with highest levels in lung and small intestine and lower levels in kidney and liver. A protein doublet is observed in human fetal brain. FIG. 2B is a digital image of two separate immunoblots of spinal cord tissue extracts from autopsy confirmed ALS and non-neurologic disease controls. Displayed are the results from 8 ALS (A) and 4 control (C) subjects. The FLJ10986 protein bands are marked on the right. Asterisk (*) denotes the presence of the risk allele of FLJ10986 within the ALS patient. The blot was stripped and probed with antibodies to actin. FLJ10986 protein data were normalized to actin levels present in each sample. FIG. 2C are bar graphs of the quantification of FLJ10986 protein levels in control and ALS spinal cord, normalized to the level of actin protein in each lane and expressed in arbitrary units (U). Error bars denote standard error of means. The left panel displays results using total FLJ10986 protein present in both immunoreactive bands for both the ALS and control subject groups. The right panel is the ratio of upper (48 kDa) to lower (45 kDa) protein bands present in ALS patients that harbor a FLJ10986 polymorphism (n=5), ALS patients lacking a FLJ10986 polymorphism (n=3), and control subjects (n=6).

FIG. 3 is Table 1. Shown are all SNPs from the initial whole genome screen that were significantly associated (p<0.05) with sALS in two independent replication populations. Columns indicate (in order) the dbSNP reference ID, the chromosome and base position of the SNP, the major allele, the allele frequency in controls (N=750), the allele frequency in sALS cases (N=766), the χ² p value for the difference between Caucasian sALS and controls (all Caucasian in all comparisons), minority ethnicities sALS case and control, the combined p value for all samples vs control, the associated gene (if any were annotated within 25 kilobase-pairs of the associated SNP on either side), and putative general biological function of the gene. The last three columns present data for the most statistically significant SNPs within the respective loci (windowing the associated SNP with 25 kb to either side in this second cohort to identify any association signal) from a completely independent study of sALS versus controls (Lovmar et al., BMC Genomics 20050; 6:35). Locus Max RS# is the rs number for the SNP at this locus with the most significant p-value (Locus p-valueₘₐₓ). The chromosome position for these SNPs are also shown. Highlighted SNPs show significance across all ALS sample sets.

FIG. 4 is Table 2. Shown are results from a comparison of three clinical subgroupings of sALS patients: A) Early Onset ALS vs Late Onset ALS; B) Female ALS vs Male ALS; C) Bulbar Onset ALS vs Limb Onset ALS. Only significant associations are shown (p<0.05). For each of these comparisons, additional comparisons to controls (N=750 in all comparisons) were performed to determine if the differences between subgroupings was primarily driven by one class. Columns indicate (in order) the dbSNP reference ID, the chromosome and base position of the SNP, the associated gene name (if any), and the overall P value for this SNP in a comparison of the entire validation series (N=766) to controls. For the comparison results, in all cases the three columns indicate the SNP allele, the allele counts in ALS:Controls, the χ² p value. The numbers of samples in each subgroup comparison are indicated.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. In the accompanying sequence listing:

SEQ ID NOs: 1-66 are the nucleotide sequences of SNPs.

SEQ ID NO: 67 is the amino acid sequence of FLJ10986.

### DETAILED DESCRIPTION

Methods are provided herein for determining the genetic predisposition of a human subject to developing any form of ALS. In several embodiments, the method determines the genetic predisposition of a subject to ALS. In additional embodiments, the method determines the genetic predisposition to a specific form of ALS in the subject (early onset, late onset, bulbar onset or limb onset).

In several embodiments, the method disclosed herein can be used to detect ALS, or can be used to detect the risk of developing ALS in a human subject.

In some embodiments, the methods utilize the detection of one or more haplotype bocks comprising a tag SNP. In additional embodiments, the methods including detecting the presence of one or more tag SNPs. The method disclosed herein can also be used to identify agents of use for treating ALS, or can be used to determine the susceptibility of a subject to treatment with a therapeutic agent of interest.

### Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of this disclosure, the following explanations of specific terms are provided:

Allele: A particular form of a genetic locus, distinguished from other forms by its particular nucleotide sequence, or one of the alternative polymorphisms found at a polymorphic site.

Amplification: To increase the number of copies of a nucleic acid molecule. The resulting amplification products are called “amplicons.” Amplification of a nucleic acid molecule (such as a DNA or RNA molecule) refers to use of a technique that increases the number of copies of a nucleic acid molecule in a sample, An example of amplification is the polymerase chain reaction (PCR), in which a sample is contacted with a pair of oligonucleotide primers under conditions that allow for the hybridization of the primers to a nucleic acid template in the sample. The primers are extended under suitable conditions, dissociated from the template, re-annealed, extended, and dissociated to amplify the number of copies of the nucleic acid. This cycle can be repeated. The product of amplification can be characterized by such techniques as electrophoresis, restriction endonuclease cleavage patterns, oligonucleotide hybridization or ligation, and/or nucleic acid sequencing.

Other examples of in vitro amplification techniques include quantitative real-time PCR; reverse transcriptase PCR (RT-PCR); real-time PCR (rt PCR); real-time reverse transcriptase PCR (rt RT-PCR); nested PCR; strand displacement amplification (see U.S. Pat. No. 5,744,311); transcription-free isothermal amplification (see U.S. Pat. No. 6,033,881); repair chain reaction amplification (see PCT Publication No. WO 90/01069); ligase chain reaction amplification (see European patent publication No. EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134), amongst others.

Amyotrophic Lateral Sclerosis (ALS): A disease also called Lou Gehrig's Disease, Maladie de Charcot or motor neuron disease. ALS is a progressive, fatal, neurodegenerative disease caused by the degeneration of motor neurons. The disorder causes muscle weakness and atrophy throughout the body as both the upper and lower motor neurons degenerate and die so that they no longer innervate the muscles. The muscles gradually weaken, atrophy and develop fasciculations (twitches) because of denervation. Eventually, the brain completely loses its ability to initiate and control voluntary movement. The disease does not necessarily debilitate the patient's mental functioning. Generally, subjects in the advanced stages of the disease retain the same memories, personality, and intelligence they had before its onset. ALS is classified into two groups, familial ALS and sporadic ALS. “Late-onset” ALS develops in individuals over the age of 60. “Early onset” ALS develops in subjects younger than 60 years of age.

The earliest symptoms of ALS may include twitching, cramping, or stiffness of muscles; muscle weakness affecting an arm or a leg; and/or slurred and nasal speech. These general complaints then develop into more obvious weakness or atrophy. The parts of the body affected by early symptoms of ALS depend on which muscles in the body are damaged first. About 75% of people experience “limb onset” ALS. Generally, in “limb-onset” ALS the symptoms first appear in a limb. In some subjects, symptoms initially affect one of the legs, and patients experience awkwardness when walking or running or they notice that they are tripping or stumbling more often. Other limb onset patients first see the effects of the disease on a hand or arm as they experience difficulty with simple tasks requiring manual dexterity such as buttoning a shirt or writing. About 25% of ALS cases are “bulbar onset” ALS. These patients first notice difficulty speaking clearly; their speech becomes garbled and slurred. Nasality and loss of volume are frequently the first symptoms; difficulty swallowing, and loss of tongue mobility follow. Eventually total loss of speech and the inability to protect the airway when swallowing are experienced.

Regardless of the part of the body first affected by the disease, muscle weakness and atrophy spread to other parts of the body as the disease progresses. Patients experience increasing difficulty moving, swallowing (dysphagia), and speaking or forming words (dysarthria). Symptoms of upper motor neuron involvement include tight and stiff muscles (spasticity) and exaggerated reflexes (hyperreflexia) including an overactive gag reflex. An abnormal reflex commonly called Babinski's sign (the large toe extends upward as the sole of the foot is stimulated) also indicates upper motor neuron damage. Symptoms of lower motor neuron degeneration include muscle weakness and atrophy, muscle cramps, and fleeting twitches of muscles that can be seen under the skin (fasciculations). Around 15-45% of patients experience pseudobulbar affect, also known as “emotional liability,” which consists of uncontrollable laughter, crying or smiling.

Only one drug is currently available for the treatment of ALS: RILUZOLE™ (Rilutek). This drug is believed to reduce damage to motor neurons by decreasing the release of glutamate. Clinical trials with ALS patients showed that riluzole prolongs survival by several months, and may have a greater survival benefit for those with a bulbar onset. The drug also extends the time before a patient needs ventilation support.

Array: An arrangement of molecules, such as biological macromolecules (such as polypeptides or nucleic acids) or biological samples (such as tissue sections), in addressable locations on or in a substrate. A “microarray” is an array that is miniaturized so as to require or be aided by microscopic examination for evaluation or analysis. Arrays are sometimes called DNA chips or biochips.

The array of molecules (“features”) makes it possible to carry out a very large number of analyses on a sample at one time. In certain example arrays, one or more molecules (such as an oligonucleotide probe) will occur on the array a plurality of times (such as twice), for instance to provide internal controls. The number of addressable locations on the array can vary, for example from a few (such as three) to at least six, at least 20, at least 25, or more. In particular examples, an array includes nucleic acid molecules, such as oligonucleotide sequences that are at least 15 nucleotides in length, such as about 15-40 nucleotides in length, such as at least 18 nucleotides in length, at least 21 nucleotides in length, or even at least 25 nucleotides in length. In one example, the molecule includes oligonucleotides attached to the array via their 5′—or 3′-end.

Within an array, each arrayed sample is addressable, in that its location can be reliably and consistently determined within the at least two dimensions of the array. The feature application location on an array can assume different shapes. For example, the array can be regular (such as arranged in uniform rows and columns) or irregular. Thus, in ordered arrays the location of each sample is assigned to the sample at the time when it is applied to the array, and a key may be provided in order to correlate each location with the appropriate target or feature position. Often, ordered arrays are arranged in a symmetrical grid pattern, but samples could be arranged in other patterns (such as in radially distributed lines, spiral lines, or ordered clusters). Addressable arrays usually are computer readable, in that a computer can be programmed to correlate a particular address on the array with information about the sample at that position (such as hybridization or binding data, including for instance signal intensity). In some examples of computer readable formats, the individual features in the array are arranged regularly, for instance in a Cartesian grid pattern, which can be correlated to address information by a computer.

Caucasian: A human racial classification traditionally distinguished by physical characteristics such as very light to brown skin pigmentation and straight to wavy or curly hair, which includes persons having origins in any of the original peoples of Europe, North Africa, or the Middle East. Popularly, the word “white” is used synonymously with “Caucasian” in North America. Such persons also retain substantial genetic similarity to natives or inhabitants of Europe, North Africa, or the Middle East. In a particular example, a Caucasian is at least 1/64 Caucasian.

Concordance: The presence of two or more loci or traits (or combination thereof) derived from the same parental chromosome. The opposite of concordance is discordance, that is, the inheritance of only one (of two or more) parental alleles and/or traits) associated with a parental chromosome.

Correlation: A correlation between a phenotypic trait and the presence or absence of a genetic marker (or haplotype or genotype) can be observed by measuring the phenotypic trait and comparing it to data showing the presence or absence of one or more genetic markers. Some correlations are stronger than others, meaning that in some instances subjects with ALS will display a particular genetic marker (i.e., 100% correlation). In other examples the correlation will not be as strong, meaning that a subject with ALS will only display a particular genetic marker 90%, 85%, 70%, 60%, 55%, or 50% of the time. In some instances, a haplotype which contains information relating to the presence or absence of multiple markers can also be correlated to a genetic predisposition to develop ALS, or the type of onset. Correlations can be described using various statistical analyses.

Decrease: Becoming less or smaller, as in number, amount, size, or intensity. In one example, decreasing the risk of a disease (such as ALS) includes a decrease in the likelihood of developing the disease by at least about 20%, for example by at least about 30%, 40%, 50%, 60%, 70%, 80%, or 90%. In another example, decreasing the risk of a disease includes a delay in the development of the disease, for example a delay of at least about six months, such as about one year, such as about two years, about five years, or about ten years.

In one example, decreasing the signs and symptoms of ALS includes decreasing the effects of the disease such as muscle weakness, difficulty moving, dysphagia, dysarthria, spasticity and/or hyperreflexia by a desired amount, for example by at least 5%, at least 10%, at least 15%, at least 20%, at least 25%, at least 30%, at least 50%, at least 75%, or even at least 90%, as compared to a response in the absence of the therapeutic composition.

DNA (deoxyribonucleic acid): DNA is a long chain polymer which comprises the genetic material of most living organisms (some viruses have genes comprising ribonucleic acid (RNA)). The repeating units in DNA polymers are four different nucleotides, each of which comprises one of the four bases, adenine, guanine, cytosine and thymine bound to a deoxyribose sugar to which a phosphate group is attached. Triplets of nucleotides (referred to as codons) code for each amino acid in a polypeptide, or for a stop signal (termination codon). The term codon is also used for the corresponding (and complementary) sequences of three nucleotides in the mRNA into which the DNA sequence is transcribed.

Unless otherwise specified, any reference to a DNA molecule is intended to include the reverse complement of that DNA molecule. Except where single-strandedness is required by the text herein, DNA molecules, though written to depict only a single strand, encompass both strands of a double-stranded DNA molecule. Thus, a reference to the nucleic acid molecule that encodes a protein, or a fragment thereof, encompasses both the sense strand and its reverse complement. Thus, for instance, it is appropriate to generate probes or primers from the reverse complement sequence of the disclosed nucleic acid molecules.

Genetic predisposition: Susceptibility of a subject to a disease, such as amyotrophic lateral sclerosis, including early onset, late onset, bulbar onset, and limb onset amyotrophic lateral sclerosis. Detecting a genetic predisposition includes detecting the presence of the disease itself, such as but not limited to an early stage of the disease process. Detecting a genetic predisposition also includes detecting the risk of developing the disease, and determining the susceptibility of that subject to developing the disease or to having a poor prognosis for the disease. Thus, if a subject has a genetic predisposition to a disease process they do not necessarily develop the disease.

Genomic target sequence: A sequence of nucleotides located in a particular region in the human genome that corresponds to one or more specific genetic abnormalities, such as a nucleotide polymorphism, a deletion, an insertion, or an amplification. The target can be for instance a coding sequence; it can also be the non-coding strand that corresponds to a coding sequence. The target can also be a non-coding sequence, such as intronic sequence. In several examples, genomic target sequences are genomic sequences of genes that encode FLJ10986, anaplastic lymphoma kinase, NADPH oxidase 4, or IQ motif containing GTPase activating protein 2.

Gene: A segment of DNA that contains the coding sequence for a protein, wherein the segment may include promoters, exons, introns, and other untranslated regions that control expression.

Genotype: An unphased 5′ to 3′ sequence of nucleotide pair(s) found at a set of one or more polymorphic sites in a locus on a pair of homologous chromosomes in an individual. “Genotyping” is a process for determining a genotype of an individual.

Haplotype: A 5′ to 3′ sequence of nucleotides found at a set of one or more polymorphic sites in a locus on a single chromosome from a single individual.

“Haplotype pair” is the two haplotypes found for a locus in a single individual. With regard to a population, haplotypes are the ordered, linear combination of polymorphisms (e.g., single nucleotide polymorphisms, SNPs) in the sequence of each form of a gene (on individual chromosomes) that exists in the population. “Haplotyping” is a process for determining one or more haplotypes in an individual and includes use of family pedigrees, molecular techniques and/or statistical inference. “Haplotype data” is the information concerning one or more of the following for a specific gene: a listing of the haplotype pairs in an individual or in each individual in a population; a listing of the different haplotypes in a population; frequency of each haplotype in that or other populations, and any known associations between one or more haplotypes and a trait.

Haplotype block: Sites of closely located SNPs which are inherited in blocks. A haplotype block includes a group of SNP locations that do not appear to recombine independently and that can be grouped together. Regions corresponding to blocks have a few common haplotypes which account for a large proportion of chromosomes. Identification of haplotype blocks is a way of examining the extent of linkage disequilibrium (LD) in the genome. The “Hap-Map” project (see the internet at the Hap-Map website) describes the mapping of haplotype blocks in the human genome.

There are programs to available on the internet for the identification of haplotype blocks, such as program HAPBLOCK™ which runs on both PC and Unix and is available from the USC website on the internet. A further program, which in addition to block identification also has visualization and selection of “tagging” SNPs is HAPLOBLOCKFINDER™, which runs interactively on the web or can be downloaded for local machine use (Unix or PC). It can be accessed at the program website available on the internet.

Hybridization: Oligonucleotides and their analogs hybridize by hydrogen bonding, which includes Watson-Crick, Hoogsteen or reversed Hoogsteen hydrogen bonding, between complementary bases. Generally, nucleic acid consists of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. “Complementary” refers to the base pairing that occurs between two distinct nucleic acid sequences or two distinct regions of the same nucleic acid sequence. For example, an oligonucleotide can be complementary to a specific genetic locus, so it specifically hybridizes with a mutant allele (and not the wild-type allele) or so that it specifically hybridizes with a wild-type allele (and not the mutant allele).

“Specifically hybridizable” and “specifically complementary” are terms that indicate a sufficient degree of complementarity such that stable and specific binding occurs between the oligonucleotide (or it's analog) and the DNA or RNA target. The oligonucleotide or oligonucleotide analog need not be 100% complementary to its target sequence to be specifically hybridizable. An oligonucleotide or analog is specifically hybridizable when binding of the oligonucleotide or analog to the target DNA or RNA molecule interferes with the normal function of the target DNA or RNA, and there is a sufficient degree of complementarity to avoid non-specific binding of the oligonucleotide or analog to non-target sequences under conditions where specific binding is desired, for example under physiological conditions in the case of in vivo assays or systems. Such binding is referred to as specific hybridization. In one example, an oligonucleotide is specifically hybridizable to DNA or RNA nucleic acid sequences including an allele of a gene, wherein it will not hybridize to nucleic acid sequences containing a polymorphism.

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ concentration) of the hybridization buffer will determine the stringency of hybridization, though wash times also influence stringency. Calculations regarding hybridization conditions required for attaining particular degrees of stringency are discussed by Sambrook et al. (ed.), Molecular Cloning: A Laboratory Manual, 2nd ed., vol. 1-3, Cold Spring Harbor Laboratory Press, Cold Spring Harbor, N.Y., 1989, chapters 9 and 11.

The following is an exemplary set of hybridization conditions and is not limiting:

Very High Stringency (Detects Sequences that Share at Least 90% Identity)

| | |
|---|---|
| Hybridization: | 5x SSC at 65° C. for 16 hours |
| Wash twice: | 2x SSC at room temperature (RT) for 15 minutes each |
| Wash twice: | 0.5x SSC at 65° C. for 20 minutes each |

High Stringency (Detects Sequences that Share at Least 80% Identity)

| | |
|---|---|
| Hybridization: | 5x-6x SSC at 65° C.-70° C. for 16-20 hours |
| Wash twice: | 2x SSC at RT for 5-20 minutes each |
| Wash twice: | 1x SSC at 55° C.-70° C. for 30 minutes each |

Low Stringency (Detects Sequences that Share at Least 50% Identity)

| | |
|---|---|
| Hybridization: | 6x SSC at RT to 55° C. for 16-20 hours |
| Wash at least twice: | 2x-3x SSC at RT to 55° C. for 20-30 minutes each. |

Isolated: An “isolated” biological component (such as a nucleic acid molecule, protein or organelle) has been substantially separated or purified away from other biological components in the cell of the organism in which the component naturally occurs, i.e., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Linkage: The association of two or more (and/or traits) at positions on the same chromosome, such that recombination between the two loci is reduced to a proportion significantly less than 50%. The term linkage can also be used in reference to the association between one or more loci and a trait if an allele (or alleles) and the trait, or absence thereof, are observed together in significantly greater than 50% of occurrences. A linkage group is a set of loci, in which all members are linked either directly or indirectly to all other members of the set.

Linkage Disequilibrium: Co-occurrence of two genetic loci (e.g., markers) at a frequency greater than expected for independent loci based on the allele frequencies. Linkage disequilibrium (LD) typically occurs when two loci are located close together on the same chromosome. When alleles of two genetic loci (such as a marker locus and a causal locus) are in strong LD, the allele observed at one locus (such as a marker locus) is predictive of the allele found at the other locus (for example, a causal locus contributing to a phenotypic trait). The linkage disequilibrium (LD) measure r² (the squared correlation coefficient) can be used to evaluate how SNPs are related on a haplotype block. For each tag SNP, the r² between that tag SNP and each additional SNP in a genotyping set can be calculated. The highest of these values is the maximum r² value, M. In several embodiments, a haplotype block can be identified SNPS that have an r² values of greater than or equal to 0.8, greater than or equal to about 0.85, greater than or equal to 0.9, or greater than or equal to 0.95 from the tag SNP.

Locus: A location on a chromosome or DNA molecule corresponding to a gene or a physical or phenotypic feature, where physical features include polymorphic sites.

Mutation: Any change of a nucleic acid sequence as a source of genetic variation. For example, mutations can occur within a gene or chromosome, including specific changes in non-coding regions of a chromosome, for instance changes in or near regulatory regions of genes. Types of mutations include, but are not limited to, base substitution point mutations (such as transitions or transversions), deletions, and insertions. Missense mutations are those that introduce a different amino acid into the sequence of the encoded protein; nonsense mutations are those that introduce a new stop codon; and silent mutations are those that introduce the same amino acid often with a base change in the third position of codon. In the case of insertions or deletions, mutations can be in-frame (not changing the frame of the overall sequence) or frame shift mutations, which may result in the misreading of a large number of codons (and often leads to abnormal termination of the encoded product due to the presence of a stop codon in the alternative frame).

Oligonucleotide: An oligonucleotide is a plurality of joined nucleotides joined by native phosphodiester bonds, between about 6 and about 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide. Functional analogs of naturally occurring polynucleotides can bind to RNA or DNA, and include peptide nucleic acid (PNA) molecules.

In several examples, oligonucleotides and oligonucleotide analogs can include linear sequences up to about 200 nucleotides in length, for example a sequence (such as DNA or RNA) that is at least 6 bases, for example at least 8, 10, 15, 20, 25, 30, 35, 40, 45, 50, 100 or even 200 bases long, or from about 6 to about 70 bases, for example about 10-25 bases, such as 12, 15 or 20 bases.

Phased: As applied to a sequence of nucleotide pairs for two or more polymorphic sites in a locus, phased means the combination of nucleotides present at those polymorphic sites on a single copy of the locus is known.

Polymorphism: A variation in a gene sequence. The polymorphisms can be those variations (DNA sequence differences) which are generally found between individuals or different ethnic groups and geographic locations which, while having a different sequence, produce functionally equivalent gene products. The term can also refer to variants in the sequence which can lead to gene products that are not functionally equivalent. Polymorphisms also encompass variations which can be classified as alleles and/or mutations which can produce gene products which may have an altered function. Polymorphisms also encompass variations which can be classified as alleles and/or mutations which either produce no gene product or an inactive gene product or an active gene product produced at an abnormal rate or in an inappropriate tissue or in response to an inappropriate stimulus. Further, the term is also used interchangeably with allele as appropriate.

Polymorphisms can be referred to, for instance, by the nucleotide position at which the variation exists, by the change in amino acid sequence caused by the nucleotide variation, or by a change in some other characteristic of the nucleic acid molecule or protein that is linked to the variation.

A “single nucleotide polymorphism (SNP)” is a single base (nucleotide) difference in a DNA sequence among individuals in a population. A tag SNP is a representative single nucleotide polymorphism (SNP) in a region of the genome with high linkage disequilibrium (the non-random association of alleles at two or more loci) that is associated with a disease, such as ALS. A tag SNP can be used to identify other SNPs, such as those with a specified r² value from the tag SNP, which are associated with a disease, such as ALS. Statistical methods to identify a tag SNP are known (see Hoperin et al., Bioinformatics 21 (suppl): i195-i203, 2005, herein incorporated by reference).

Probes and primers: A probe comprises an isolated nucleic acid capable of hybridizing to a target nucleic acid. A detectable label or reporter molecule can be attached to a probe or primer. Typical labels include radioactive isotopes, enzyme substrates, co-factors, ligands, chemiluminescent or fluorescent agents, haptens, and enzymes. Methods for labeling and guidance in the choice of labels appropriate for various purposes are discussed, for example in Sambrook et al. (In Molecular Cloning: A Laboratory Manual, CSHL, New York, 1989) and Ausubel et al. (In Current Protocols in Molecular Biology, John Wiley & Sons, New York, 1998).

In a particular example, a probe includes at least one fluorophore, such as an acceptor fluorophore or donor fluorophore. For example, a fluorophore can be attached at the 5′—or 3′-end of the probe. In specific examples, the fluorophore is attached to the base at the 5′-end of the probe, the base at its 3′-end, the phosphate group at its 5′-end or a modified base, such as a T internal to the probe.

Probes are generally at least 15 nucleotides in length, such as at least 15, at least 16, at least 17, at least 18, at least 19, least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50 at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, or more contiguous nucleotides complementary to the target nucleic acid molecule, such as 20-70 nucleotides, 20-60 nucleotides, 20-50 nucleotides, 20-40 nucleotides, or 20-30 nucleotides.

Primers are short nucleic acid molecules, for instance DNA oligonucleotides 10 nucleotides or more in length, which can be annealed to a complementary target nucleic acid molecule by nucleic acid hybridization to form a hybrid between the primer and the target nucleic acid strand. A primer can be extended along the target nucleic acid molecule by a polymerase enzyme. Therefore, primers can be used to amplify a target nucleic acid molecule.

The specificity of a primer increases with its length. Thus, for example, a primer that includes 30 consecutive nucleotides will anneal to a target sequence with a higher specificity than a corresponding primer of only 15 nucleotides. Thus, to obtain greater specificity, probes and primers can be selected that include at least 15, 20, 25, 30, 35, 40, 45, 50, 55, 60, 65, 70 or more consecutive nucleotides. In particular examples, a primer is at least 15 nucleotides in length, such as at least 15 contiguous nucleotides complementary to a target nucleic acid molecule. Particular lengths of primers that can be used to practice the methods of the present disclosure include primers having at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 45, at least 50, at least 55, at least 60, at least 65, at least 70, or more contiguous nucleotides complementary to the target nucleic acid molecule to be amplified, such as a primer of 15-70 nucleotides, 15-60 nucleotides, 15-50 nucleotides, or 15-30 nucleotides.

Primer pairs can be used for amplification of a nucleic acid sequence, for example, by PCR, real-time PCR, or other nucleic-acid amplification methods known in the art. An “upstream” or “forward” primer is a primer 5′ to a reference point on a nucleic acid sequence. A “downstream” or “reverse” primer is a primer 3′ to a reference point on a nucleic acid sequence. In general, at least one forward and one reverse primer are included in an amplification reaction.

Nucleic acid probes and primers can be readily prepared based on the nucleic acid molecules provided herein. It is also appropriate to generate probes and primers based on fragments or portions of these disclosed nucleic acid molecules, for instance regions that encompass the identified polymorphisms of interest. PCR primer pairs can be derived from a known sequence by using computer programs intended for that purpose such as Primer (Version 0.5, © 1991, Whitehead Institute for Biomedical Research, Cambridge, Mass.) or PRIMER EXPRESS® Software (Applied Biosystems, AB, Foster City, Calif.).

Sample: A sample, such as a biological sample, is a sample obtained from a subject. As used herein, biological samples include all clinical samples useful for detection of amyotrophic lateral sclerosis in subjects, including, but not limited to, cells, tissues, and bodily fluids, such as: blood; derivatives and fractions of blood, such as serum; extracted galls; biopsied or surgically removed tissue, including tissues that are, for example, unfixed, frozen, fixed in formalin and/or embedded in paraffin; tears; milk; skin scrapes; surface washings; urine; sputum; cerebrospinal fluid; prostate fluid; pus; or bone marrow aspirates. In a particular example, a sample includes blood obtained from a human subject, such as whole blood or serum. In another particular example, a sample includes buccal cells, for example collected using a swab or by an oral rinse.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods. This homology is more significant when the orthologous proteins or cDNAs are derived from species which are more closely related (such as human and mouse sequences), compared to species more distantly related (such as human and C. elegans sequences).

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site.

BLASTN is used to compare nucleic acid sequences, while BLASTP is used to compare amino acid sequences. To compare two nucleic acid sequences, the options can be set as follows: -i is set to a file containing the first nucleic acid sequence to be compared (such as C:\seq1.txt); -j is set to a file containing the second nucleic acid sequence to be compared (such as C:\seq2.txt); -p is set to blastn; -o is set to any desired file name (such as C:\output.txt); -q is set to −1; -r is set to 2; and all other options are left at their default setting. For example, the following command can be used to generate an output file containing a comparison between two sequences: C:\B12seq -i c:\seq1.txt -j c:\seq2.txt -p blastn -o c:\output.txt -q-1-r 2.

To compare two amino acid sequences, the options of B12seq can be set as follows: -i is set to a file containing the first amino acid sequence to be compared (such as C:\seq1.txt); -j is set to a file containing the second amino acid sequence to be compared (such as C:\seq2.txt); -p is set to blastp; -o is set to any desired file name (such as C:\output.txt); and all other options are left at their default setting. For example, the following command can be used to generate an output file containing a comparison between two amino acid sequences: C:\B12seq c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1154 nucleotides is 75.0 percent identical to the test sequence (i.e., 1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (that is, 15÷20*100=75).

One indication that two nucleic acid molecules are closely related is that the two molecules hybridize to each other under stringent conditions, as described above. Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. Such homologous nucleic acid sequences can, for example, possess at least 60%, at least 70%, at least 80%, at least 90%, at least 95%, at least 98%, or at least 99% sequence identity determined by this method. An alternative (and not necessarily cumulative) indication that two nucleic acid sequences are substantially identical is that the polypeptide which the first nucleic acid encodes is immunologically cross reactive with the polypeptide encoded by the second nucleic acid. One of skill in the art will appreciate that the particular sequence identity ranges are provided for guidance only.

Subject: Living multi-cellular vertebrate organisms, a category that includes human and non-human mammals (such as laboratory or veterinary subjects).

Therapeutically effective amount: An amount of a therapeutic agent that alone, or together with one or more additional therapeutic agents, induces the desired response, such as decreasing the risk of developing ALS or decreasing the signs and symptoms of ALS. Ideally, a therapeutically effective amount provides a therapeutic effect without causing a substantial cytotoxic effect in the subject. The preparations disclosed herein are administered in therapeutically effective amounts.

In one example, a desired response is to prevent the development of ALS. In another example, a desired response is to delay the development or progression of ALS, for example, by at least about three months, at least about six months, at least about one year, at least about two years, at least about five years, or at least about ten years. In another example, a desired response is to decrease the signs and symptoms of ALS, such as the neurological symptoms in the limbs or associated with speaking. In general, a therapeutically effective amount of a composition administered to a human subject will vary depending upon a number of factors associated with that subject, for example the overall health of the subject, the condition to be treated, or the severity of the condition. A therapeutically effective amount of a composition can be determined by varying the dosage of the product and measuring the resulting therapeutic response. The therapeutically effective amount can be dependent on the source applied, the subject being treated, the severity and type of the condition being treated, and the manner of administration.

Wild-type: A genotype that predominates in a natural population of organisms that do not have a disease process, such as ALS. A wild-type genotype differs from mutant forms.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### Methods for Detecting a Genetic Predisposition to ALS

Methods for determining the genetic predisposition of a subject to ALS are provided herein. These methods include methods for determining the genetic predisposition to any form of ALS, as well as specific methods for determining the genetic predisposition of a subject to early onset, late onset, bulbar onset and limb onset ALS. The methods can be used to detect any form of ALS in the subject (early onset, late onset, bulbar onset or limb onset). The method can also be used to detect the risk of developing ALS.

In some embodiments the methods include obtaining a sample including nucleic acids from a human subject of interest, and analyzing for the presence of haplotype blocks including a tag SNP in these nucleic acids. Biological samples include all clinical samples useful for detection of amyotrophic lateral sclerosis in subjects, including, but not limited to, cells, tissues, and bodily fluids, such as: blood; derivatives and fractions of blood, such as serum; extracted galls; biopsied or surgically removed tissue, including tissues that are, for example, unfixed, frozen, fixed in formalin and/or embedded in paraffin; tears; milk; skin scrapes; surface washings; urine; sputum; cerebrospinal fluid; prostate fluid; pus; or bone marrow aspirates. In a particular example, a sample includes blood obtained from a human subject, such as whole blood or serum. In another particular example, a sample includes buccal cells, for example collected using a swab or by an oral rinse. In additional embodiments, the method includes analyzing DNA sequence data previously obtained from the subject of interest.

Generally, these methods utilize the detection of one or more haplotype bocks comprising a tag SNP. In several embodiments, the methods including detecting the presence of one or more tag SNPs themselves. In some embodiments, identifying the presence of one or more tag SNPs, or haplotype blocks including these SNPs, determines the effectiveness of a therapeutic agent for treatment of the subject.

In one example, a method for detecting a genetic predisposition to ALS in a human subject by detecting the presence of one or more halotype blocks including a tag SNP. Each haplotype block is identified by (and includes) a tag SNP. Thus, detecting the presence of the haplotype block can include detecting a SNP with r² value of greater than about 0.8, about 0.85, about 0.9 or about 0.95 from a tag SNP.

Specific haplotype blocks of use to identifying a genetic predisposition to ALS include the following tag SNPs: rs6690993, wherein position 59416003 is a G; rs6700125, wherein position 59414818 is a T; rs7074175, wherein position 20556984 is a T; rs4827700 wherein position 145052081 is a G; rs6036180 wherein position 22627977 is an A; rs2836061 wherein position 38247104 is a C; rs2279605 wherein position 55611622 is an A; rs4756063 wherein position 33822142 is a G; rs11018623 wherein position 88837360 is a G; rs4629724 wherein position 121250591 is a T; rs470-4336 wherein position 75899375 is a G; rs5970919 wherein position 22639221 is an A; rs5929816 wherein position 136099981 is an A; rs2279607 wherein position 55611764 is a T; rs7003876 wherein position 1135748 is a T; rs988213 wherein position 42378965 is an A; rs2036535 wherein position 28775126 is a T; rs5925683 wherein position 22629374 is a C; rs10499100 wherein position 121250044 is a T; rs1172149 wherein position 201956415 is a T; rs3810715 wherein position 150555188 is a G; rs13036957 wherein position 41255110 is a G; rs752257 wherein position 22630289 is a G; rs17027230 wherein position 102537848 is a C; rs757863 wherein position 77316032 is an A; rs10740320 wherein position 70840449 is a G; rs4263905 wherein position 145052983 is a T; rs10942784 wherein position 75889806 is an A; rs10809959 wherein position 13497924 is a C; rs10762294 wherein position 70840387 is a C; rs1466471 wherein position 61478245 is a G; rs3744477 wherein position 40183199 is a T; rs10748358 wherein position 421-49850 is a T; rs12119273 wherein position 61655314 is a G; rs10834819 wherein position 25821137 is a G; rs10506228 wherein position 42150219 is a T; rs12995017 wherein position 205046522 is an A; rs945699 wherein position 224400054 is a G; rs1554914 wherein position 150549225 is a T; rs4287603 wherein position 2722492 is a G; rs1027615 wherein position 41998556 is an A; rs666481 wherein position 10010682 is a C; rs1447830 wherein position 74695861 is a C; rs12473579 wherein position 203030073 is a G; rs905080 wherein position 41995195 is a G; rs2205545 wherein position 150677351 is an A; rs3771150 wherein position 102519369 is a C; rs1891592 wherein position 148367576 is an A; rs3749870 wherein position 155646464 is a G; rs12279181 wherein position 25819399 is an A; rs11172457 wherein position 56752884 is a G; rs733281 wherein position 41264461 is a T; rs4819840 wherein position 18096320 is an A; rs4491817 wherein position 18097369 is a G; rs1314625 wherein position 26844530 is a C; rs4516412 wherein position 203029371 is a G; rs879012 wherein position 957788 is a G; rs27628 wherein position 50266128 is a T; rs276915 wherein position 26853979 is an A; rs38271 wherein position 14080271 is a C; rs276916 wherein position 26854159 is a C; rs7772593 wherein position 106451750 is a T; rs7937375 wherein position 21698795 is an A; rs27248 wherein position 50268304 is an A; rs4622670 wherein position 29357853 is a G; or rs7818421 wherein position 8328291 is a C. The presence of one or more of these haplotype blocks determines the genetic predisposition to amyotrophic lateral sclerosis in the human subject. The method can include detecting the presence of at least five, at least ten, at least twenty, at least thirty, at least forty or at least fifty different haplotype bocks, each including a different one of the tag SNPs. The groups of haplotype blocks can be in any combination, of at least five, ten twenty, thirty, forty or fifty different haplotype blocks.

The method can also include detecting one of more of the tag SNPs themselves. Thus, the method can include detecting at least at least five, at least ten, at least twenty, at least thirty, at least forty or at least fifty of the SNPs. The groups of tag SNPs can be in any combination, of at least five, ten twenty, thirty, forty or fifty different tag SNPs. Detection of all of the tag SNPs can also be used to detect a genetic predisposition to ALS.

With regard to the SNPs, the SNPs are identified by name. The exact sequence of the SNP can be determined from the database of SNPs available at the NCBI website (Entrez SNP, dbSNP build 128). The “position” is the location in the genome of the SNP, referring to the nucleotide position from the p-terminus of the chromosome in the human genome, see the NCBI SNP website, available on the internet. Sequence information for each of the tag SNPs listed above is provided in the following table:

| | | | ALS | SEQ |
|---|---|---|---|---|
| | Encoded | | risk | ID |
| SNP | Protein | Sequence | allele | NO: |
| rs6690993 | FLJ10986 | aaaacaaagttattaggcgga | G | 1 |
| | | gaaag[A/G]catgccaatga | | |
| | | cttgagcacttaa | | |
| rs6700125 | FLJ10986 | ggtttctgtcatagctgagat | T | 2 |
| | | tccat[C/T]gactatgagca | | |
| | | acttgcagacaggg | | |
| rs7074175 | PLXDC2 | cgtaagttatctgggtggaca | T | 3 |
| | | gtggtgcca[C/T]tgaatag | | |
| | | gaaatacaggaagagaaaggg | | |
| | | gg | | |
| rs4827700 | | agtgagagggaagcacgactt | G | 4 |
| | | tccggcagc[A/G]tccatct | | |
| | | gttgtcttgttgcttgtctgc | | |
| | | ca | | |
| rs6036180 | FOXA2 | gccaattgagcatacaatctg | A | 5 |
| | | atgacctct[A/G]tgcttcc | | |
| | | aattaagttcagtctcccaac | | |
| | | tc | | |
| rs2836061 | | ggctgcgcactgcacctgcca | C | 6 |
| | | aactgtgcg[C/T]tccaggc | | |
| | | tctaggctctgtttttgctgg | | |
| | | cc | | |
| rs2279605 | CGNL1 | taaatgttgatatacagttcg | A | 7 |
| | | tctagcacc[A/G]tttccag | | |
| | | gctctaggcgtacaactgggt | | |
| | | aa | | |
| rs4756063 | | tttgactttttcttaacctca | G | 8 |
| | | taaatactt[A/G]gttctca | | |
| | | gaatgtgaacatgtaaatagt | | |
| | | aa | | |
| rs11018623 | NOX4 | ttgttttgtttgatcattatt | G | 9 |
| | | aatccataa[C/G]cttcaca | | |
| | | agcatcagttacttcaggtgt | | |
| | | tt | | |
| rs4629724 | C6orf170 | ccagtagtttgcgtaaattat | T | 10 |
| | | gtttctgtt[G/T]tttgtgt | | |
| | | gtttctatgctctctcctcat | | |
| | | tt | | |
| rs4704336 | IQGAP2 | gttttaaaatgctctaattcc | G | 11 |
| | | aatatgtag[A/G]atgttag | | |
| | | catccaatgatgacaatgtaa | | |
| | | tt | | |
| rs5970919 | | gttcagacaccattattcaag | A | 12 |
| | | caataatca[A/G]tttggca | | |
| | | acacgggaacttaccggaatc | | |
| | | aa | | |
| rs5929816 | | ataatctagtcccagcaagtt | A | 13 |
| | | tcttctca[A/G]cttttgat | | |
| | | gaatctggagaataatagaca | | |
| | | t | | |
| rs2279607 | CGNL1 | tgaatacaaattaattatctt | T | 14 |
| | | tgaaactgc[C/T]gctgagt | | |
| | | gttctgaatcctcttcccaaa | | |
| | | at | | |
| rs7003876 | Spliced | aaaacctccctattgttttga | T | 15 |
| | EST | aatgacaac[A/T]acagctt | | |
| | | cgtcgtggggcgtcacgcatg | | |
| | | tt | | |
| rs988213 | LOXHD1 | tacctgagcaaagctcctgaa | A | 16 |
| | | caactctac[A/C]atgcctg | | |
| | | gagtgcctcatactgagagta | | |
| | | tc | | |
| rs2036535 | ACCN1 | agcatcaaactgaatagaatt | T | 17 |
| | | gttgtttgt[C/T]cagatca | | |
| | | gccatctgtgttttacagagc | | |
| | | aa | | |
| rs5925683 | | aacgttactgcaatgtctaat | C | 18 |
| | | ctgaactta[C/G]gccatta | | |
| | | tgctctttgacctggaaagtg | | |
| | | ta | | |
| rs10499100 | C6orf170 | taatcttcttgcttcttttgc | T | 19 |
| | | ccaagacta[C/T]gcttctt | | |
| | | gcaacctggcattgtttacgg | | |
| | | at | | |
| rs1172149 | TMCC2 | tttagctgccatgaaaggcag | T | 20 |
| | | tccagccac[C/T]gcagttc | | |
| | | tgtctcaggtagaaacaaaga | | |
| | | ca | | |
| rs3810715 | FATE1 | agttcttctgccagggacatt | G | 21 |
| | | tccatctcc[A/C]ccttggt | | |
| | | gttgggagggcctcctgccat | | |
| | | ca | | |
| rs13036957 | PTPRT | tgccatcatgtagcttacaat | G | 22 |
| | | ctggtggag[A/G]agactgg | | |
| | | acaaagagaaaggggtaacaa | | |
| | | cc | | |
| rs752257 | | tgtccttataagaaaggaaaa | G | 23 |
| | | tgtggacac[A/G]tgcagac | | |
| | | acagaaggacagcatcacgtg | | |
| | | tg | | |
| rs17027230 | | ctttgccaaaattcaaggtca | C | 24 |
| | | actgaaaaa[C/T]gccccat | | |
| | | ttaacctctgattgtaccacc | | |
| | | cc | | |
| rs757863 | MAGI2 | gttccattaacatggatactg | A | 25 |
| | | agagcatcc[A/G]tgagtac | | |
| | | aggagacatgagacatgggcc | | |
| | | ta | | |
| rs10740320 | TACR2 | aaaagtgactccatcttggat | G | 26 |
| | | gccactccc[A/G]tgtgttg | | |
| | | acttccgattcgccccagtct | | |
| | | tg | | |
| rs4263905 | | ttcgtcttcaagtaagtgaca | T | 27 |
| | | agctattcc[G/T]ccccaaa | | |
| | | tctgttcacagtgcttgacac | | |
| | | ag | | |
| rs10942784 | IQGAP2 | tatcatacttcggggagcttt | A | 28 |
| | | tatttttgc[A/C]tcctgcc | | |
| | | ccttgctcccaattgtcaact | | |
| | | ta | | |
| rs10809959 | | tgtatgtagtgcattattttt | C | 29 |
| | | atccacttt[C/T]tccccct | | |
| | | cctagaggcaggcatgccatg | | |
| | | aa | | |
| rs10762294 | TACR2 | ctgatgagctgcaggctgggg | C | 30 |
| | | actgactcc[C/T]cggcagg | | |
| | | tctgcagtgggaacctgtgga | | |
| | | ga | | |
| rs1466471 | | aaactgcgtctgaccacagaa | G | 31 |
| | | tcattactc[A/G]ccaccct | | |
| | | gagagctggagccatgagcta | | |
| | | gt | | |
| rs3744477 | DBF4B | aacatttaaagagacaactct | T | 32 |
| | | ttaaatgca[C/T]ggacgca | | |
| | | gagaagggtgagaagaatcac | | |
| | | gg | | |
| rs10748358 | ADAMTS20 | ttcttaggagttaaaaacttg | T | 33 |
| | | aacatcttc[C/T]tagcatt | | |
| | | taaggtagatgtcctaacaac | | |
| | | ta | | |
| rs12119273 | NFIA | ggacatcagcatatgaattgg | G | 34 |
| | | ggaaggccc[A/G]aacactc | | |
| | | caccgataacagtaacatggt | | |
| | | tt | | |
| rs10834819 | | atagaaatgagtcacaaggat | G | 35 |
| | | gttgtgaag[A/G]taagaat | | |
| | | atcattggttgagaaaggtct | | |
| | | gt | | |
| rs10506228 | ADAMTS20 | attctacaattcatgcttgca | T | 36 |
| | | tgttcagtt[G/T]tttagtg | | |
| | | gatcatcatcttattatatgt | | |
| | | tt | | |
| rs12995017 | | ccataacaaccagatgccgtg | A | 37 |
| | | tatttgatc[A/T]gttaaat | | |
| | | cattcccaaacagttaaaata | | |
| | | tg | | |
| rs945699 | Zfp709 | ttcctccttcccacttgcggt | G | 38 |
| | | gtgattcca[A/G]gagctct | | |
| | | aggcctctctgggccttcagg | | |
| | | tg | | |
| rs1554914 | | gaataggggctggaggatgtt | T | 39 |
| | | ttctgaaat[G/T]ctctgga | | |
| | | gccacctcctccttgggactt | | |
| | | cc | | |
| rs4287603 | GARNL4 | atataagctgctttgaaacaa | G | 40 |
| | | agaccgttg[A/G]ttacaga | | |
| | | ggcttatctcaggagggggct | | |
| | | tt | | |
| rs1027615 | | cagaagttcgtaaataattta | A | 41 |
| | | gaacctaaa[A/G]tatgcta | | |
| | | tttacaatgcttatggattct | | |
| | | ag | | |
| rs666481 | | ctgatctgcctcctcctgcct | C | 42 |
| | | ctaagccat[C/T]gtccagt | | |
| | | tgacattattctcttgagatc | | |
| | | cg | | |
| rs1447830 | | aggagctgcaataagcccaat | C | 43 |
| | | ggataggag[C/T]ggtccct | | |
| | | gctcgctgatgatgttcatat | | |
| | | ta | | |
| rs12473579 | | ggacctatgccaggaactatt | G | 44 |
| | | ctatatgat[A/G]caaatgc | | |
| | | aacagtgaacaaaatgaacca | | |
| | | ag | | |
| rs905080 | | acacatgtgccctgtctgcca | G | 45 |
| | | tcttgagct[C/G]gcaataa | | |
| | | gaatagaagtaactatgaatt | | |
| | | tt | | |
| rs2205545 | | tatagaaatgacaagctgatt | A | 46 |
| | | ctaaaatac[A/G]tatgaaa | | |
| | | agacaaaaactgttgcatgta | | |
| | | ta | | |
| rs3771150 | IL18RAP | tccatagattatcaatgggtg | C | 47 |
| | | atggaggct[C/T]accgggg | | |
| | | ctaagggtcatgttgacatca | | |
| | | ga | | |
| rs1891592 | TUFT1 | tcccaatctgatgaaggcctg | A | 48 |
| | | gcttatggc[A/G]cagccag | | |
| | | agtccagtctacaacagaagg | | |
| | | ga | | |
| rs3749870 | TIAM2 | taccccgtagtagtttaaaga | G | 49 |
| | | ttccaaaac[G/T]cacagga | | |
| | | aagttgaaaggcctcaggctg | | |
| | | tg | | |
| rs12279181 | | ttaaaacttgctttctctatg | A | 50 |
| | | tatgttacc[A/G]gagatat | | |
| | | cgagttactgtaaacatccct | | |
| | | at | | |
| rs11172457 | | ttggggcctctccagtcacct | G | 51 |
| | | ttcttaagc[A/G]tccacaa | | |
| | | aaaaggtaacttctagtgcct | | |
| | | tt | | |
| rs733281 | PTPRT | acctggcctagaatcttaaca | T | 52 |
| | | cgcttctcc[C/T]gggcagg | | |
| | | agtgtaatgagtaaagggagc | | |
| | | tc | | |
| rs4819840 | | caccccgtgcccttattgccc | A | 53 |
| | | actgaaggt[A/G]ctcgcgc | | |
| | | ggcaccaactgcactggcttc | | |
| | | ct | | |
| rs4491817 | | tgcactggtgtctggatgtgc | G | 54 |
| | | tcagaggcc[C/G]cctgggt | | |
| | | ccccaggcctctgggacaagg | | |
| | | cc | | |
| rs1314625 | DSC3 | agagaatttacaggctttcct | C | 55 |
| | | attaattaa[C/T]gagatta | | |
| | | actaatcccattaatctttag | | |
| | | ga | | |
| rs4516412 | | agctggcaacattcaatgtca | G | 56 |
| | | tcctccttc[A/G]ctgataa | | |
| | | ctcagctgccttgtgctgaga | | |
| | | gg | | |
| rs879012 | | cccattgtacatcagcctgcc | G | 57 |
| | | acagggaac[A/G]actatcc | | |
| | | tgtccccattgccagaaccct | | |
| | | gc | | |
| rs27628 | PARP8 | gaccttgagagataaggaaga | T | 58 |
| | | agacatgca[G/T]ttccctt | | |
| | | ttctaggtttgagcccagaag | | |
| | | tc | | |
| rs276915 | DSC3 | catcaaacaatacaagaaatg | A | 59 |
| | | caggagaac[A/G]attctcg | | |
| | | gaggaagtaaacactattgag | | |
| | | gt | | |
| rs38271 | DGKB | ccagcaattgggctagtcatc | C | 60 |
| | | tttctgctc[C/T]acgtggc | | |
| | | atgacaagatcacttggcatc | | |
| | | tt | | |
| rs276916 | DSC3 | taatttcaaccaccatctaga | C | 61 |
| | | tactgatga[C/T]ttctggg | | |
| | | tagacatttctagtcatgatc | | |
| | | tc | | |
| rs7772593 | | gccacctaggagtcctctcct | T | 62 |
| | | tcccttggc[C/T]catcctt | | |
| | | caaccacacatcagcgtccaa | | |
| | | aa | | |
| rs7937375 | NELL1 | tccaaatgaatgggacacaaa | A | 63 |
| | | acattattt[A/T]atattgg | | |
| | | tcagacttgtatcatttgcta | | |
| | | cg | | |
| rs27248 | PARP8 | accaagtaatgatggtcagac | A | 64 |
| | | caagttagt[A/G]aaaaggt | | |
| | | catttgatagtcgtaacccca | | |
| | | ct | | |
| rs4622670 | ALK | acccctctcctcccaggacgg | G | 65 |
| | | cagcagggc[A/G]ctcaccg | | |
| | | aatgagggtgatgtttttccg | | |
| | | cg | | |
| rs7818421 | | ttggaaattggggttctcaga | C | 66 |
| | | tgccccacc[C/T]cattcca | | |
| | | catttatctattttatatttc | | |
| | | ag | | |

In this table, the “risk” allele identifies the tag SNP that can be used to detect ALS. The “wild-type” allele is a different allele not associated with ALS. In the sequences provided above, the notation “[X/Y]” is used, wherein one of X or Y is the risk allele and one of X or Y is the wild-type allele. For each sequence, the allele associated with ALS (the “risk” allele) is listed in the column entitled “ALS risk allele.”

In several embodiments, subjects that have a haplotype block associated with the wild-type allele are not genetic pre-disposed to developing ALS. These subjects do not have ALS and/or have a low risk for developing ALS.

In several embodiments, detecting the presence of a haplotype block comprises detecting a single nucleotide polymorphism with an r² value of about 0.8 or greater from rs6690993, wherein position 59416003 is a G; rs6700125, wherein position 59414818 is a T; rs7074175, wherein position 20556984 is a T; rs4827700 wherein position 145052081 is a G; rs6036180 wherein position 22627977 is an A; rs2836061 wherein position 38247104 is a C; rs2279605 wherein position 55611622 is an A; rs4756063 wherein position 33822142 is a G; rs11018623 wherein position 88837360 is a G; rs4629724 wherein position 121250591 is a T; rs470-4336 wherein position 75899375 is a G; rs5970919 wherein position 22639221 is an A; rs5929816 wherein position 136099981 is an A; rs2279607 wherein position 55611764 is a T; rs7003876 wherein position 1135748 is a T; rs988213 wherein position 42378965 is an A; rs2036535 wherein position 28775126 is a T; rs5925683 wherein position 22629374 is a C; rs10499100 wherein position 121250044 is a T; rs1172149 wherein position 201956415 is a T; rs3810715 wherein position 150555188 is a G; rs13036957 wherein position 41255110 is a G; rs752257 wherein position 22630289 is a G; rs17027230 wherein position 102537848 is a C; rs757863 wherein position 77316032 is an A; rs10740320 wherein position 70840449 is a G; rs4263905 wherein position 145052983 is a T; rs10942784 wherein position 75889806 is an A; rs10809959 wherein position 13497924 is a C; rs10762294 wherein position 70840387 is a C; rs1466471 wherein position 61478245 is a G; rs3744477 wherein position 40183199 is a T; rs10748358 wherein position 421-49850 is a T; rs12119273 wherein position 61655314 is a G; rs10834819 wherein position 25821137 is a G; rs10506228 wherein position 42150219 is a T; rs12995017 wherein position 205046522 is an A; rs945699 wherein position 224400054 is a G; rs1554914 wherein position 150549225 is a T; rs4287603 wherein position 2722492 is a G; rs1027615 wherein position 41998556 is an A; rs666481 wherein position 10010682 is a C; rs1447830 wherein position 74695861 is a C; rs12473579 wherein position 203030073 is a G; rs905080 wherein position 41995195 is a G; rs2205545 wherein position 150677351 is an A; rs3771150 wherein position 102519369 is a C; rs1891592 wherein position 148367576 is an A; rs3749870 wherein position 155646464 is a G; rs12279181 wherein position 25819399 is an A; rs11172457 wherein position 56752884 is a G; rs733281 wherein position 41264461 is a T; rs4819840 wherein position 18096320 is an A; rs4491817 wherein position 18097369 is a G; rs1314625 wherein position 26844530 is a C; rs4516412 wherein position 203029371 is a G; rs879012 wherein position 957788 is a G; rs27628 wherein position 50266128 is a T; rs276915 wherein position 26853979 is an A; rs38271 wherein position 14080271 is a C; rs276916 wherein position 26854159 is a C; rs7772593 wherein position 106451750 is a T; rs7937375 wherein position 21698795 is an A; rs27248 wherein position 50268304 is an A; rs4622670 wherein position 29357853 is a G; or rs7818421 wherein position 8328291 is a C.

In one example, when the method includes detecting the presence of the haplotype block comprising rs6690993, wherein position 59416003 is a G, the method can include detecting the presence of one or more of rs835380, rs12758288, rs3738172, rs11207416, rs12139438, rs333668, rs333666, rs12402265, rs12752853, rs11207431, rs6587850, rs835378, rs12145786, rs17118876, rs11207422, rs11207409, rs1475629, rs11207426, rs7531917, rs12730750, rs7547161, rs7521970, rs7554924 or a combination thereof. In another example, when the method includes detecting the presence of the haplotype block comprising rs6700125, wherein position 59414818 is a T, the method can include detecting the presence of rs835376, rs12758288, rs12405063, rs11207416, rs588302, rs168002, rs10749717, rs12739904, rs7542194, rs11207431, rs1373646, rs7526027, rs1475629, rs7547161, rs12730750, rs7554924, rs7521970, rs17118876, rs12145786, rs835378, rs11207409, rs11207422, rs7531917, or a combination thereof. In another example, when the method includes detecting the presence of the haplotype block comprising rs7074175, wherein position 20556984 is a T, the method includes detecting the presence of rs10828012, rs11593311, rs1041555, rs10827974, rs7919470, rs7907088, rs10827980, rs3817405, rs12772292, rs1855085, rs11011868, rs11011893, rs4338440, rs7917594, rs2778984, rs985477, rs10764214, rs2358870, rs11011826, rs7070780, rs6482113, rs10764218, rs11011878, rs12259741, rs7895512, rs11011877, rs11011872, rs11011874, rs12774800 or a combination thereof. In another example, when the method includes detecting the presence of the haplotype block comprising rs7074175, wherein position 20556984 is a T, the method includes detecting the presence of rs10828012, rs11593311, rs1041555, rs10827974, rs7919470, rs7907088, rs10827980, rs3817405, rs12772292, rs1855085, rs11011868, rs11011893, rs4338440, rs7917594, rs2778984, rs985477, rs10764214, rs2358870, rs11011826, rs7070780, rs6482113, rs10764218, rs11011878, rs12259741, rs7895512, rs11011877, rs11011872, rs11011874, rs12774800 or a combination thereof. In another example, when the method includes detecting the presence of the haplotype block comprising rs4827700 wherein position 145052081 is a G, the method includes detecting the presence of rs5966123, rs4240056, rs910618, rs6525671, rs12014291, rs7876742, rs5920035, rs12389980, rs12559830, rs5920062, rs4399062, rs17311536 or a combination thereof. In another example, when the method includes detecting the presence of the haplotype block comprising rs6036180 wherein position 22627977 is an A, the method includes detecting the presence of rs4603850, rs2148920, rs6113803, rs6113762, rs6036187, rs6048344, rs11906222, rs8117625, rs6048350, rs4813468, rs996669, rs874525, rs6048351, rs4813469, rs11907005, rs2424460, rs6082783 or a combination thereof. In another example, when the method includes detecting the presence of the haplotype block comprising rs2836061 wherein position 38247104 is a C, the method comprises detecting the presence of rs9984628, rs6517438, rs2409950, rs2836048, rs2186343, rs762147, rs2836112, rs9974219, rs2836067, rs7275707, rs2836080, rs2836128, rs2836058, rs1029001, rs2836074, rs914150, rs2257130, rs2836108, rs2836079, rs11088411, rs928765, rs3827199, rs4817904, rs1892567, rs6517442, rs2836082, rs4816585, rs3787870, rs2836106, rs8129919, rs974975 or a combination thereof. In another example, when the method includes detecting the presence of the haplotype block comprising rs2279605 wherein position 55611622 is an A, the method includes detecting the presence of rs2932208, rs2934429, rs937254, rs2451186, rs6493934, rs1908202, rs2934417, rs1296301, rs1292363, rs2934431, rs744318, rs12593015, rs7172968, rs1620402, rs11857299, rs2131730, rs1280380, rs1280408, rs11857629, rs2934449, rs7181031, rs2470360, rs1280409, rs1280376, rs2279605, rs7168095, rs17820365, rs12591554, rs477-4967, rs7179379, rs17820299, rs8034838, rs477-4948, rs2414512, rs477-4952, rs1018268, rs1995989, rs1908199, rs1908188, rs17820461, rs745998, rs1280419, rs11071331, rs1873993, rs12915561, rs17820383, rs7169081, rs1814313, rs1567619, rs744379, rs1107114, rs11632793, rs1280398, rs477-4940, rs1297111, rs7179813, rs2635383, rs1280400, rs11632868, rs766103, rs477-4963, rs7165557 or a combination thereof. In another example, when the method includes detecting the presence of the haplotype block comprising rs4756063 wherein position 33822142 is a G the method includes detecting the presence of rs939804, rs12807373, rs11032385, rs10836123, rs17038, rs746481, rs2038602, rs3758641, rs1176359, rs11032383, rs3758638, rs2901376, rs4007, rs1885524, rs7941248, rs1533800, rs10836130, rs7119163, rs4756077, rs11032412, rs941940, rs3781578, rs3758642, rs941941, rs3781575, rs11032401, rs3824848, rs7946026, rs10836131, rs10836132, rs3758640, rs10836127, rs10836126, rs12285414, rs12790679, rs4756063, rs10742308 or a combination thereof. In another example, when the method includes detecting the presence of the haplotype block comprising rs11018623 wherein position 88837360 is a G, the method includes detecting the presence of rs3017883, rs317155, rs2202151, rs957140, rs319023, rs7396916, rs317127, rs585197, rs3019011, rs10830263, rs672549, rs12799930, rs2289123, rs497279, rs12276991 or a combination thereof. In another example, when the method includes detecting the presence of the haplotype block comprising rs4629724 wherein position 121250591 is a T the method includes detecting the presence of rs10484956, rs4629724, rs9374980, rs7747737, rs9374988, rs2050736, rs2817961, rs2789074, rs925812, rs12192195, rs2817930, rs9374974 or a combination thereof. In another example, when the method includes detecting the presence of the haplotype block comprising rs470-4336 wherein position 75899375 is a G, the method includes detecting the presence of rs2455227, rs461273, rs10044688, rs470-4347, rs382669, rs13153728, rs13173162, rs470-4336, rs2431348, rs12109754, rs1038920, rs10056943, rs10038589, rs9293688, rs460562, rs2069680, rs6872396, rs3797385, rs3797412, rs12697857, rs470-4338, rs3736394, rs470-4318, rs950643, rs6453230, rs10035948, rs9293692, rs32947, rs7727095, rs1501788, rs6875519, rs10045331, rs2069656, rs1697845, rs10474483, rs7734540, rs17748322, rs7711045, rs458059, rs17652073, rs2059222, rs13187591, rs7710225, rs2069658, rs10942782, rs13168171, rs6869765, rs3797437, rs3797446, rs2455232, rs3797390, rs253096, rs2069693, rs7707762 or a combination thereof. In another example, when the method includes detecting the presence of the haplotype block comprising rs5970919 wherein position 22639221 is an A, the method includes detecting the presence of rs5926086, rs7889437, rs5970884, rs6528187, rs972377, rs7050046, rs5925668, rs5926096, rs5926070, rs5926097, rs1974517, rs5926151, rs2040774, rs6629507, rs12353647, rs5926095, rs6528191, rs5970664, rs1034726, rs964467, rs5970663, rs5970937, rs2214520, rs5970939, rs6528223, rs11094872, rs5970907, rs5970943, rs5926063, rs6528208, rs7057694, rs5926103, rs6629492, rs2214519, rs1859286 or a combination thereof. In another example, when the method includes detecting the presence of the haplotype block comprising rs5929816 wherein position 136099981 is an A, the method includes detecting the presence of rs1547320, rs5975880, rs2840664, rs4127903, rs2743903, rs1412555, rs2743905, rs5929816 or a combination thereof. In another example, when the method includes detecting the presence of the haplotype block comprising rs2279607 wherein position 55611764 is a T, the method includes detecting the presence of rs2932208, rs2934429, rs937254, rs2451186, rs6493934, rs1908202, rs2934417, rs1296301, rs1292363, rs2934431, rs744318, rs12593015, rs7172968, rs1620402, rs11857299, rs2131730, rs1280380, rs1280408, rs11857629, rs2934449, rs7181031, rs2470360, rs1280409, rs1280376, rs2279605, rs7168095, rs17820365, rs12591554, rs477-4967, rs7179379, rs17820299, rs8034838, rs477-4948, rs2414512, rs477-4952, rs1018268, rs1995989, rs1908199, rs1908188, rs17820461, rs745998, rs1280419, rs11071331, rs1873993, rs12915561, rs17820383, rs7169081, rs1814313, rs1567619, rs744379, rs1107114, rs11632793, rs1280398, rs477-4940, rs1297111, rs7179813, rs2635383, rs1280400, rs11632868, rs766103, rs477-4963, rs7165557 or a combination thereof. In another example, when the method includes detecting the presence of the haplotype block comprising rs7003876 wherein position 1135748 is a T the method includes detecting the presence of rs7009597, rs10097829, rs13272049, rs12678079, rs13275958, rs17815788, rs2084803, rs7832117, rs760179, rs12543247, rs4311679, rs12675380, rs10113837, rs11136360, rs4548203, rs4976875, rs12549181, rs1451886, rs4141052, rs10283028, rs4875987, rs12547340, rs10111719, rs11136379, rs11779225, rs10099450, rs9693703, rs7834689, rs7822337, rs13257422, rs11988553, rs2251870, rs9314423, rs7829754, rs13267567, rs7006250, rs11784898, rs13261832, rs1470777, rs12681748, rs7003876, rs13273765, rs6995458, rs6981899, rs7823268, rs12548097, rs13270217, rs4735993, rs6558423, rs4272406, rs10046782, rs7843593, rs10104676, rs11775878, rs1562923, rs7814902, rs4735996, rs11136377, rs4295683 rs11779163, rs7827676, rs11136373 or a combination thereof. In another example, when the method includes detecting the presence of the haplotype block comprising rs988213 wherein position 42378965 is an A, the method includes detecting the presence of rs328123, rs1450428, rs9944943, rs12456713, rs16939675, rs11082536, rs9946967, rs426303, rs328174, rs1349626, rs3744859, rs16978566, rs7228531, rs17689183, rs4890672, rs732109, rs7232709, rs1606889, rs12454466, rs1376079, rs328144, rs328173, rs4890667, rs7235623, rs10468983, rs2156282, rs328172, rs1462981, rs11662957, rs12458980, rs9957285, rs328125, rs1376080, rs9965681, rs1450425, rs4133965, rs1812125, rs4890674, rs101940, rs328189, rs328190, rs986117, rs17766830, rs12962116, rs101941, rs744744, rs644468 or a combination thereof. In another example, when the method includes detecting the presence of the haplotype block comprising rs2036535 wherein position 28775126 is a T the method includes detecting the presence of rs12453548, rs12601163, rs1827650, rs13341516, rs12602581, rs9894658, rs12452051, rs11652534, rs11080213, rs17249146, rs10853155, rs11656544, rs17184873, rs320633, rs1394393, rs7223348, rs6505339, rs12941575, rs1504581, rs4462650, rs2036535, rs8075499, rs320637, rs3935891, rs4795799, rs1504578, rs412602, rs1995526, rs7225320, rs7213646, rs1354491, rs16968444, rs1394395, rs1007035, rs12941035, rs8068295, rs7215745, rs9891235, rs9914954, rs7222667, rs733136, rs17185084, rs8064308, rs4589606, rs12945313, rs4795800, rs2036537, rs63954, rs2347152, rs4366752, rs17836785, rs12450310, rs12453418, rs7207490, rs4305127, rs1504571, rs12942506, rs1021891, rs4795769, rs479-4947, rs1354492, rs7210939 or a combination thereof. In another example, when the method includes detecting the presence of the haplotype block comprising rs5925683 wherein position 22629374 is a C, the method includes detecting the presence of rs4366252, rs5926102, rs7064630, rs5970645, rs7064507, rs5970899, rs7050046, rs5926096, rs2189488, rs5926097, rs4828924, rs5926152, rs1859285, rs6633624, rs12353647, rs1007490, rs4828937, rs6629492, rs2214520, rs5970663, rs5970939, rs985852, rs7880430, rs5970937, rs964467, rs5970907, rs1034726, rs2214519, rs5970664, rs5970943, rs6528208, rs6528223, rs1859286, rs5926103, rs7057694, rs11094872, rs5926063 or a combination thereof. In another example, when the method includes detecting the presence of the haplotype block comprising rs10499100 wherein position 121250044 is a T the method includes detecting the presence of rs10484956, rs4629724, rs9374980, rs7747737, rs9374988, rs2050736, rs2817961, rs2789074, rs925812, rs12192195, rs2817930, rs9374974 or a combination thereof. In another example, when the method includes detecting the presence of the haplotype block comprising rs1172149 wherein position 201956415 is a T the method includes detecting the presence of rs1172111, rs10900472, rs11240443, rs2290265, rs11240449, rs10494860, rs11240410, rs1106201, rs7533637, rs1172132, rs12022806, rs4951173, rs1779410, rs11240451, rs4951201, rs12067235, rs4951182, rs9661015, rs10157145, rs2864859, rs10751435, rs12760299, rs17345837 or a combination thereof. In another example, when the method includes detecting the presence of the haplotype block comprising rs3810715 wherein position 150555188 is a G, the method includes detecting the presence of rs5924997, rs4553055, rs12852212, rs5970086, rs10745191, rs5924985, rs5970114, rs12861962, rs1554913, rs5925000, rs722351, rs5925043, rs5924979, rs1123319, rs5970097, rs12839777, rs950254, rs5925030, rs3893333, rs12860832, rs4828577, rs5924989, rs5924658, rs6627473, rs5970118, rs5970123, rs5925006, rs10482211, rs6627452, rs3761541, rs5924654, rs5925018, rs5925038, rs12839220, rs5924662, rs5924683, rs7054854, rs5925023 or a combination thereof. In another example, when the method includes detecting the presence of the haplotype block comprising rs13036957 wherein position 41255110 is a G, the method includes detecting the presence of rs2425602, rs927058, rs11697714, rs6030670, rs6030675, rs6030669, rs2205773, rs6016963, rs1572925, rs6030661, rs4812681, rs6065582, rs2425610, rs3092409, rs11086869, rs2867657, rs6072984, rs6072990, rs2425607, rs2092105, rs2205772, rs10485698, rs13041343, rs6030703, rs1539035, rs6072981, rs11086863, rs2425588 or a combination thereof. In another example, when the method includes detecting the presence of the haplotype block comprising rs752257 wherein position 22630289 is a G the method includes detecting the presence of rs6036197, rs2148921, rs2179859, rs6075934, rs6515254, rs2424472, rs11697574, rs8125753, rs6048350, rs996669, rs11907005, rs4813469, rs6048354, rs4813468, rs6048351, rs874525, rs6082846, rs11477053, rs2424460, rs8115044, rs6048352, rs6082783 or a combination thereof. In another example, when the method includes detecting the presence of the haplotype block comprising rs17027230 wherein position 102537848 is a C the method includes detecting the presence of rs1035127, rs3213732, rs4851611, rs4851016, rs3771171, rs4241211, rs7561351, rs10172553, rs2310302, rs2140316, rs6543135, rs11692304, rs1974675, rs759382, rs1468788, rs7605606, rs4851005, rs4851601, rs11690532 or a combination thereof. In another example, when the method includes detecting the presence of the haplotype block comprising rs757863 wherein position 77316032 is an A, the method includes detecting the presence of rs848451, rs2903949, rs2023950, rs13243925, rs2428918, rs11761224, rs3807727, rs886595, rs2960457, rs3807786, rs6954104, rs6466037, rs6943424, rs6465932, rs2428932, rs3807721, rs3779323, rs848461, rs848458, rs10953392, rs3779308, rs3807757, rs3807707, rs719313, rs3807769, rs1211911, rs735406, rs11508506, rs6958027, rs10953456, rs4729938, rs3779331, rs1465221, rs7789223, rs4590377, rs7805185, rs2906510, rs2074646, rs848464, rs7777605, rs11975124, rs7791394, rs757863, rs7799860, rs3807743, rs17614508, rs2428929, rs2428927, rs2428928, rs3779330, rs3807736, rs848467, rs4727608, rs6971524, rs12668675, rs848472, rs3779347, rs757865, rs1205283, rs3779317 or a combination thereof. In another example, when the method includes detecting the presence of the haplotype block comprising rs10740320 wherein position 70840449 is a G the method includes detecting the presence of rs10823384, rs10823354, rs10823357, rs12784882, rs4745987, rs1367517, rs1652804, rs6480403, rs7917338, rs4746849, rs3829183, rs1227942, rs10762290, rs749105, rs1624453, rs906215, rs10998730, rs10823356, rs2305196, rs10823369, rs2305197, rs1665581, rs7097078, rs749107, rs4746846, rs10998805, rs1238357, rs2290020, rs2084274, rs12355201, rs2278745, rs2015803, rs1864589, rs10998716, rs7909192, rs10823343, rs7098301, rs10762288, rs10998740 or a combination thereof. In another example, when the method includes detecting the presence of the haplotype block comprising rs4263905 wherein position 145052983 is a T the method includes detecting the presence of rs9792792, rs6525662, rs1934238, rs5966146, rs12014291, rs7876742, rs5920035, rs4399062, rs12559830, rs17311536, rs12389980, rs5920062 or a combination thereof. In another example, when the method includes detecting the presence of the haplotype block comprising rs10942784 wherein position 75889806 is an A, the method includes detecting the presence of rs2455227, rs461273, rs10044688, rs458994, rs7711417, rs13173162, rs6869692, rs4326119, rs388058, rs3797376, rs6888854, rs460562, rs2202113, rs2069680, rs4145111, rs3797412, rs3822528, rs10077289, rs3797410, rs470-4320, rs3736394, rs2069664, rs2068434, rs10045331, rs10942782, rs32947, rs13187591, rs17748322, rs2455219, rs7727095, rs2069656, rs9293692, rs10474483, rs4452539, rs1501788, rs2059222, rs458059, rs2069658, rs7707762, rs3797435, rs2455232, rs13168171, rs7711045, rs2069693, rs10035948, rs17652073, rs6875519, rs7710225, rs3797390 or a combination thereof. In another example, when the method includes detecting the presence of the haplotype block comprising rs10809959 wherein position 13497924 is a C, the method includes detecting the presence of rs10491757, rs10961159, rs977580, rs1156273, rs12238169, rs2184230, rs10756475, rs1889297, rs933034, rs7853368, rs12553986, rs1953175, rs7857061, rs10121700, rs1324188, rs10961147, rs12235656, rs10809959, rs1556576, rs1543714, rs2018555, rs12000433, rs1324190, rs4741307, rs10733261, rs10809958, rs2225173, rs12352391, rs3737150, rs1408319, rs10961170 or a combination thereof. In another example, when the method includes detecting the presence of the haplotype block comprising rs10762294 wherein position 70840387 is a C, the method includes detecting the presence of rs10823391, rs10823354, rs1227938, rs12784882, rs464-4560, rs1367517, rs1052179, rs7072268, rs4746849, rs3829183, rs7917338, rs3829185, rs749108, rs906215, rs1624453, rs1227942, rs5030922, rs4746846, rs749107, rs2015803, rs1238357, rs2305197, rs10823356, rs7909192, rs10998716, rs1665581, rs7097078, rs10762288, rs906216, rs2084274, rs10998740, rs12355201, rs2290020, rs10823343, rs7098301, rs2305196, rs2278745, rs10998805, rs1864589 or a combination thereof. In another example, when the method includes detecting the presence of the haplotype block comprising rs1466471 wherein position 61478245 is a G, the method includes detecting the presence of rs10504312, rs1552071, rs10957135, rs7007407, rs1383239, rs7464602, rs11998308, rs7001157, rs2086393, rs10088581, rs11775891, rs11776538, rs6981694, rs931139, rs922610 or a combination thereof. In another example, when the method includes detecting the presence of the haplotype block comprising rs3744477 wherein position 40183199 is a T, the method includes detecting the presence of rs1558085, rs10853009, rs2070605, rs9907151, rs6503402, rs11871429, rs8064331, rs12450654, rs7213960, rs10852995 or a combination thereof. In another example, when the method includes detecting the presence of the haplotype block comprising rs10748358 wherein position 421-49850 is a T, the method includes detecting the presence of rs11182163, rs4768500, rs10880516, rs6582464, rs6582473, rs10736012, rs10880524, rs7960952, rs12306994, rs11182133, rs10785430, rs10880480 or a combination thereof. In another example, when the method includes detecting the presence of the haplotype block comprising rs12119273 wherein position 61655314 is a G, the method includes detecting the presence of rs7512200, rs2886084, rs4915745, rs2474383, rs2365257, rs2474384, rs12132826, rs2207791, rs1933302, rs12727960, rs10493307, rs9436640, rs11207774, rs2092867, rs4132542, rs12040431, rs2207792, rs1884367, rs4915748, rs2474379, rs7513561, rs2246514, rs10489908, rs2152981, rs10889221, rs2499533, rs6587933 or a combination thereof. In another example, when the method includes detecting the presence of the haplotype block comprising rs10834819 wherein position 25821137 is a G, the method includes detecting the presence of rs11028926, rs930459, rs10767451, rs17308502, rs11028971, rs1036755, rs10834823, rs7104555, rs7930648, rs12806413, rs1441518, rs1908162, rs10767474, rs1441493, rs327491, rs12418699, rs10834832, rs10501023, rs1441483, rs2033977, rs7948650, rs1441491, rs1372269, rs1532286, rs2859991, rs11823887, rs10834836, rs11029028, rs10834805, rs10219359 or a combination thereof. In another example, when the method includes detecting the presence of the haplotype block comprising rs10506228 wherein position 42150219 is a T, the method includes detecting the presence of rs11182163, rs4768500, rs10880516, rs6582464, rs6582473, rs10736012, rs10880524, rs7960952, rs12306994, rs11182133, rs10785430, rs10880480 or a combination thereof. In another example, when the method includes detecting the presence of the haplotype block comprising rs12995017 wherein position 205046522 is an A, the method includes detecting the presence of rs10211485, rs6435238, rs10206538, rs10207985, rs6741142, rs10180613, rs6435227, rs10180781, rs4675440, rs4673291, rs13390018, rs7608404, rs11695187, rs7590649, rs11690070, rs7572741, rs10932058, rs759880, rs9288348, rs6435231, rs11684723, rs10183904, rs2353847, rs6747433, rs6760812, rs726833, rs6435236, rs1981913, rs6435232, rs7599379, rs11894121 or a combination thereof. In another example, when the method includes detecting the presence of the haplotype block comprising rs945699 wherein position 224400054 is a G, the method includes detecting the presence of rs697763, rs4128390, rs2527614, rs6659918, rs878811, rs1582114, rs12121588, rs12748472, rs10127943, rs1891410, rs4341357, rs6673695, rs945699, rs12046421, rs1009658, rs6426478, rs10799458, rs10916243, rs849901, rs20488 or a combination thereof. In another example, when the method includes detecting the presence of the haplotype block comprising rs1554914 wherein position 150549225 is a T, the method includes detecting the presence of rs5924675, rs12857408, rs6627176, rs5970081, rs5970114, rs10745191, rs12861962, rs1123319, rs1554913, rs4449925, rs5925008, rs13441013, rs926039, rs12839777, rs722351, rs3761540, rs12556787, rs5924985, rs5925023, rs10482211, rs6627473, rs5925006, rs3761541, rs3893333, rs5924658, rs6627452, rs5924662, rs12839220, rs5970123, rs4828577, rs5925038, rs5970118, rs5924683, rs5924989, rs5925018, rs7054854, rs5924654, rs12860832 or a combination thereof. In another example, when the method includes detecting the presence of rs4287603 wherein position 2722492 is a G, the method includes detecting the presence of rs7209248, rs4790385, rs8071247, rs2317462, rs7222386, rs2027998, rs17835077, rs2028000, rs4790098, rs11652404, rs4790099, rs8076548, rs1079530, rs11652853, rs12951927, rs9907411, rs9904506, rs1476461, rs8069911, rs9895492, rs8080301, rs11652546, rs8075141, rs9747501, rs6502574, rs6502566, rs11869792, rs12936006, rs11867235, rs9915468, rs9905703, rs9910861, rs9674744, rs12937985, rs12450785, rs9889673, rs7406606, rs1036911, rs9913366, rs715662, rs1476460, rs4790104, rs9909521, rs4790102, rs11656002, rs7226198, rs735176, rs12950923, rs1124040, rs6502555, rs9890608, rs8072995, rs123059, rs2317469, rs9902403, rs7207754, rs9909561, rs4790393, rs8072316, rs12946748, rs11653110, rs11869022 or a combination thereof. In another example, when the method includes detecting the presence of the haplotype block comprising rs1027615 wherein position 41998556 is an A, the method includes detecting the presence of rs2019623, rs10880468, rs6582456, rs12812173, rs11181996, rs11182055, rs12369483, rs10880440, rs7970905, rs12306994, rs10880480, rs1849777, rs2134067, rs10748354, rs1317608 or a combination thereof. In another example, when the method includes detecting the presence of the haplotype block comprising rs666481 wherein position 10010682 is a C, the method includes detecting the presence of rs483972, rs607712, rs8085302, rs985784, rs29129, rs678194, rs502547, rs571890, rs687997, rs29063, rs29189, rs12969307, rs591814, rs587316, rs7229522, rs29056, rs8091187, rs1985509, rs526989, rs571021, rs9945100, rs3069, rs8087137, rs1231580, rs522276, rs29033, rs586409, rs29072, rs11660506, rs29069, rs4797397, rs9945403, rs495116, rs9950398, rs636407, rs552242, rs29191, rs29029, rs658513, rs610573, rs8089099, rs618909, rs29031, rs29047, rs593676 or a combination thereof. In another example, when the method includes detecting the presence of the haplotype block comprising rs1447830 wherein position 74695861 is a C, the method includes detecting the presence of rs4677417, rs9874773, rs13092046, rs6549597, rs11128393, rs7432669, rs12054115, rs7430259, rs9310301, rs11710153, rs17711625, rs6549601, rs7429855, rs17012697, rs11923571, rs1374878, rs13078817, rs1374866, rs7639846, rs935523, rs9830820, rs13073838, rs9819617, rs6799372 or a combination thereof. In another example, when the method includes detecting the presence of the haplotype block comprising rs12473579 wherein position 203030073 is a G, the method includes detecting the presence of rs10084427, rs1996270, rs7583801, rs6758247, rs6435143, rs4303700, rs6758561 or a combination thereof. In another example, when the method includes detecting the presence of the haplotype block comprising rs905080 wherein position 41995195 is a G, the method includes detecting the presence of rs2062654, rs871880, rs1027616, rs17093054, rs11181996, rs17521729, rs11181988, rs10748354, rs10880480, rs7970905, rs2134067, rs1849777, rs12306994, rs1317608 or a combination thereof. In another example, when the method includes detecting the presence of the haplotype block comprising rs2205545 wherein position 150677351 is an A, the method includes detecting the presence of rs5925072, rs964181, rs6526045, rs5970102, rs6627187, rs5925043, rs5925054, rs13441013, rs5925062, rs941401, rs2205549, rs4828577, rs12389766, rs6627480, rs5970123, rs12843815, rs5970118, rs6627473, rs5925038, rs6526041, rs5925023, rs7054854 or a combination thereof.

In another example, when the method includes detecting the presence of the haplotype block comprising rs3771150 wherein position 102519369 is a C, the method includes detecting the presence of rs6717915, rs10208196, rs2075188, rs3771171, rs2871474, rs2008157, rs6543144, rs3771166, rs1523204, rs6719196, rs2287037, rs6543146, rs4851604, rs7558013, rs759382, rs1468788, rs1921622, rs11690532, rs10208293, rs7605606, rs4851005, rs4851601 or a combination thereof. In another example, when the method includes detecting the presence of the haplotype block comprising rs1891592 wherein position 148367576 is an A, the method includes detecting the presence of rs12747990, rs1891593, rs1196357, rs11204848, rs1891588, rs17640598, rs9651181, rs3790506 or a combination thereof. In another example, when the method includes detecting the presence of the haplotype block comprising rs3749870 wherein position 155646464 is a G, the method includes detecting the presence of rs1886576, rs9371867, rs162971, rs6911945, rs17812980, rs226314, rs17086050, rs950994, rs1408756, rs9478626, rs428-447, rs9397804, rs1032141, rs12717192, rs1334687, rs13219130, rs9885806, rs162977, rs7747147, rs6909568, rs17739072, rs9480075, rs7756965, rs9322504, rs912722, rs6557410, rs324361, rs6933801, rs9478621, rs7770051, rs1485754, rs2352423, rs1108371, rs9371371, rs9384304, rs9480091, rs6909739, rs17086041, rs9397797, rs7767306, rs9371870, rs12525523, rs1032143, rs10081066, rs9478616, rs10872715, rs9384297, rs730536, rs9397793, rs721101, rs324368, rs12663896 or a combination thereof. In another example, when the method includes detecting the presence of the haplotype block comprising rs12279181 wherein position 25819399 is an A, the method includes detecting the presence of rs10834798, rs10160290, rs11028965, rs10734365, rs2165511, rs1372268, rs10767471, rs12576724, rs12785467, rs1348167, rs11028988, rs6484176, rs10767474, rs327491, rs10834788, rs10400315, rs10834851, rs10834832, rs2033979, rs1441483, rs1372270, rs1372269, rs1441491, rs2859991, rs10834836, rs10219359, rs1532286, rs11823887, rs10834805, rs11029028 or a combination thereof. In another example, when the method includes detecting the presence of the haplotype block comprising rs11172457 wherein position 56752884 is a G, the method includes detecting the presence of rs1506888, rs2653867, rs2221320, rs871907, rs10735876, rs4403838, rs2036451, rs11613082, rs1502815, rs2939817, rs435-4731, rs1502814, rs9738727, rs2733449, rs265579, rs12422977, rs12426565, rs1109125 or a combination thereof. In another example, when the method includes detecting the presence of the haplotype block comprising rs733281 wherein position 41264461 is a T, the method includes detecting the presence of rs11699547, rs2425602, rs4812672, rs10485700, rs734981, rs6030669, rs6030675, rs1572926, rs2867602, rs2425599, rs2185591, rs6093784, rs6030660, rs2425610, rs1539035, rs2205772, rs2211285, rs10485698, rs2425594, rs6030703, rs11086869, rs13041343, rs6130333, rs6072990, rs6072981, rs2425607, rs2092105, rs3092130, rs11086863, rs2867657 or a combination thereof. In another example, when the method includes detecting the presence of the haplotype block comprising rs4819840 wherein position 18096320 is an A, the method includes detecting the presence of rs5748402, rs5993830, rs9606113, rs9618708, rs6518580, rs9617842, rs5748412, rs13053792, rs12159686, rs2238777, rs4819516, rs5748401, rs9606160, rs5748366, rs5992465, rs5748410, rs5748370, rs6518569, rs5993794, rs1005133, rs1541326, rs4819835, rs1123656, rs5748391, rs7285337, rs4819523, rs5993810, rs9606112, rs2157731, rs8136246, rs7291533, rs9618678, rs5748414, rs5748407, rs11089305, rs12483887, rs9618649, rs4819837, rs4819833, rs7291584, rs2097599, rs5748406, rs5748362, rs739374, rs5993820, rs17209532, rs2157732, rs8135854, rs11089296, rs7292279, rs1978060, rs5748396, rs5748433, rs1473107, rs885988 or a combination thereof. In another example, when the method includes detecting the presence of the haplotype block comprising rs4491817 wherein position 18097369 is a G, the method includes detecting the presence of rs4819832, rs5993830, rs9808864, rs1468089, rs9306226, rs7291613, rs737869, rs6518580, rs4819840, rs13057911, rs5993834, rs1015939, rs4819516, rs5748366, rs5748407, rs5748370, rs739374, rs7291533, rs5748433, rs4819523, rs4819833, rs5748396, rs4819837, rs8136246, rs5993794, rs7291584, rs5748391, rs7285337, rs16984538, rs9618678, rs11089296, rs11089305, rs1123656, rs5748406, rs2157731, rs5748410, rs1005133, rs6518569, rs4819835, rs2097599, rs5993810, rs1978060, rs1473107, rs8135854, rs885988, rs5748414, rs5993820, rs5748362, rs1541326, rs7292279, rs5992465, rs2157732, rs12483887, rs9606112 or a combination thereof. In another example, when the method includes detecting the presence of the haplotype block comprising rs1314625 wherein position 26844530 is a C, the method includes detecting the presence of rs276912, rs3910339, rs1789068, rs1790695, rs12605921, rs276930, rs1595356, rs1623381, rs1369364, rs1260720, rs1313586, rs276938, rs2640847, rs3910498, rs1658097, rs7240980, rs276943, rs175776, rs1612474, rs1260719, rs4799559, rs5004530, rs1790690, rs1313578, rs8765, rs1790683, rs2850323, rs9961958, rs276910, rs9959549, rs1313595rs10502553rs10502557rs12456649 or a combination thereof rs4516412 wherein position 203029371 is a G the method includes detecting the presence of rs6735994, rs12994463, rs6435146, rs6435143, rs6758561, rs6758247, rs4303700 or a combination thereof. In another example, when the method includes detecting the presence of the haplotype block comprising rs879012 wherein position 957788 is a G, the method includes detecting the presence of rs6118784, rs6140946, rs6086545, rs805639, rs6118727, rs6108322, rs562081, rs6140734, rs879013, rs4816165, rs1884113, rs6140882, rs6077759, rs2223961, rs6140951, rs502716, rs534577, rs7361530, rs6077826, rs6087024, rs479848, rs13043111, rs6086845, rs579591, rs1854569, rs6056135, rs530913, rs530652, rs2064733, rs6056778, rs6056182, rs4816169, rs761857, rs504507, rs578505, rs550408, rs480789, rs6077478, rs8120339, rs6087091, rs533608, rs6056646, rs6074148, rs6056558, rs11087853, rs11698234, rs6056178, rs564744, rs6077755, rs4813031, rs6140963, rs6056615, rs6131084 or a combination thereof. In another example, when the method includes detecting the presence of the haplotype block comprising rs27628 wherein position 50266128 is a T, the method includes detecting the presence of rs27283, rs4443373, rs3991902, rs10055437, rs27863, rs6898567, rs10054150, rs4866022, rs10461650, rs152839, rs250238, rs27785, rs11957654, rs12110116, rs27964, rs250230, rs27252, rs11746623, rs3846499, rs13172653, rs635788, rs27905, rs588023, rs27267, rs27580, rs1363846 or a combination thereof. In another example, when the method includes detecting the presence of the haplotype block comprising rs276915 wherein position 26853979 is an A, the method includes detecting the presence of rs276915, rs12326186, rs1602895, rs1658101, rs1595356, rs1389367, rs276931, rs1658102, rs1260720, rs1369364, rs2640847, rs276937, rs7229311, rs1658097, rs16961025, rs190681, rs1658096, rs2276373, rs1790689, rs8765, rs4799559, rs10502553, rs2850323, rs1260719, rs1790690, rs276910, rs5004530, rs1313595, rs1790683, rs1313578, rs9961958, rs9959549, rs10502557, rs12456649 or a combination thereof. In another example, when the method includes detecting the presence of the haplotype block comprising rs38271 wherein position 14080271 is a C, the method includes detecting the presence of rs6461073, rs38285, rs10244768, rs12112152, rs17168106, rs6977358, rs17167991, rs2215034, rs6949550, rs7458793, rs13309076, rs11766474, rs17767576, rs7801010, rs12699600, rs12673837, rs17167942, rs6461069, rs2099281, rs6955394, rs6461076, rs38291, rs7780524, rs17168080, rs6974135, rs12699607, rs7799696, rs17712036, rs976760, rs12699601, rs7800027, rs17765925, rs6962879, rs10950513, rs6954527, rs2108203, rs6971997, rs1859732, rs7780422, rs12667568, rs6955524, rs1016804, rs10251211, rs2190385, rs38274, rs6971533, rs10240586, rs17775102, rs17774495 or a combination thereof. In another example, when the method includes detecting the presence of the haplotype block comprising rs276916 wherein position 26854159 is a C, the method includes detecting the presence of rs276915, rs12326186, rs1602895, rs1658101, rs1595356, rs1389367, rs276931, rs1658102, rs1260720, rs1369364, rs2640847, rs276937, rs7229311, rs1658097, rs16961025, rs190681, rs1658096, rs2276373, rs1790689, rs8765, rs4799559, rs10502553, rs2850323, rs1260719, rs1790690, rs276910, rs5004530, rs1313595, rs1790683, rs1313578, rs9961958, rs9959549, rs10502557, rs12456649 or a combination thereof. In another example, when the method includes detecting the presence of the haplotype block comprising rs7772593 wherein position 106451750 is a T, the method includes detecting the presence of rs1567268, rs12195148, rs4946704, rs6900659, rs4946705, rs9384590, rs10457137, rs4945737, rs9386492, rs1876560, rs7760913, rs6924145, rs1876563, rs11152938, rs11152943, rs2895618, rs11152927, rs2400135, rs4568493, rs1007664, rs4530899, rs1876552, rs9398056, rs7772224, rs1876555, rs7746082, rs6934413, rs7452412, rs4472384, rs4946713, rs9386496, rs12202386, rs4945734, rs12199117, rs7748283, rs12526107, rs4245527, rs7766016, rs7746706, rs4620150, rs4946700, rs9386490, rs1508355, rs9480604, rs1876557, rs1355021, rs6568420, rs6901669, rs12195588, rs9386485, rs7749121, rs7738222, rs9480610, rs10457138, rs2400136, rs9320132, rs6938669, rs4245529, rs1107166, rs12526093, rs9399953, rs9320141 or a combination thereof.

In another example, when the method includes detecting the presence of the haplotype block comprising rs7937375 wherein position 21698795 is an A, the method includes detecting the presence of rs10833615, rs10833584, rs7924938, rs764949, rs10766845, rs1557438, rs10833642, rs11026216, rs10833580, rs11026257, rs1380510, rs1945544, rs10741889, rs2000949, rs7951149, rs990339, rs1459886, rs10766848, rs1459887 or a combination thereof.

In another example, when the method includes detecting the presence of the haplotype block comprising rs27248 wherein position 50268304 is an A, the method includes detecting the presence of rs27283, rs4443373, rs3991902, rs10055437, rs27863, rs6898567, rs10054150, rs4866022, rs10461650, rs152839, rs250238, rs27785, rs11957654, rs12110116, rs27964, rs250230, rs27252, rs11746623, rs3846499, rs13172653, rs635788, rs27905, rs588023, rs27267, rs27580, rs1363846 or a combination thereof. In another example, when the method includes detecting the presence of the haplotype block comprising rs4622670 wherein position 29357853 is a G, the method includes detecting the presence of rs13386033, rs11127204, rs4666168, rs3100232, rs2123443, rs2290366, rs12997783, rs11127208, rs10432708, rs17783899, rs11885445, rs6708752, rs876748, rs7425108, rs6547915, rs1358514, rs2697336, rs4666183, rs17749904, rs1073319, rs12997875, rs2339379, rs7561367, rs3100227, rs6744522, rs2293563, rs10178180, rs1728829, rs7599783, rs11127207, rs7572088, rs1728827, rs2879455, rs3768674, rs13014227, rs4666184, rs7599255, rs1104870, rs1358513, rs12998728, rs6750752, rs12619049, rs11683248, rs4666178, rs3100244, rs1869264, rs6723311, rs2339469, rs10432706 or a combination thereof. In another example, when the method includes detecting the presence of the haplotype block comprising rs7818421 wherein position 8328291 is a C, the method includes detecting the presence of rs4240617, rs2979172, rs4500083, rs2979240, rs2921076, rs7000407, rs2976887, rs4840982, rs2980742, rs2976926, rs2980772, rs6990504, rs2979202, rs11777086, rs2976921, rs2921023, rs2921008, rs10087493, rs10503376, rs2921040, rs17617027, rs2921028, rs7005904, rs13256028, rs2976872, rs2976956, rs2980754, rs4840971, rs4840344, rs13273161, rs17607190, rs6601694, rs2979164, rs2976940, rs2979181, rs13277477, rs4840974, rs13280051, rs2976868, rs2920986, rs2945865, rs7833103, rs2921094, rs10089767, rs4840341, rs2979144, rs13280304, rs7001723, rs2979153, rs1971412, rs2979166, rs4375003, rs17150353, rs17669452, rs4840939, rs6997361, rs12546760, rs2921098, rs6981750, rs2976876 or a combination thereof.

Thus, in several embodiments, detecting the presence of a haplotype block comprises detecting a single nucleotide polypmorphism with an r² value of 0.8 or greater, 0.85 or greater, 0.9 or greater, or 0.95 or greater from one or more tag SNPs. One of skill in the art can readily identify additional single nucleotide polymorphisms with an r² value of about 0.8 or greater, about 0.85 or greater, about 0.9 or greater, or about 0.95 or greater from one of the tag SNPs of use in the methods disclosed herein.

In several embodiments, the method includes detecting the presence of one or more tag SNPs. Thus, the method can include detecting the presence of at least five, at least ten, at least twenty, at least thirty, at least forty, at least fifty tag SNPs themselves, wherein the tag SNPs are rs6690993, wherein position 59416003 is a G; rs6700125, wherein position 59414818 is a T; rs7074175, wherein position 20556984 is a T; rs4827700 wherein position 145052081 is a G; rs6036180 wherein position 22627977 is an A; rs2836061 wherein position 38247104 is a C; rs2279605 wherein position 55611622 is an A; rs4756063 wherein position 33822142 is a G; rs11018623 wherein position 88837360 is a G; rs4629724 wherein position 121250591 is a T; rs470-4336 wherein position 75899375 is a G; rs5970919 wherein position 22639221 is an A; rs5929816 wherein position 136099981 is an A; rs2279607 wherein position 55611764 is a T; rs7003876 wherein position 1135748 is a T; rs988213 wherein position 42378965 is an A; rs2036535 wherein position 28775126 is a T; rs5925683 wherein position 22629374 is a C; rs10499100 wherein position 121250044 is a T; rs1172149 wherein position 201956415 is a T; rs3810715 wherein position 150555188 is a G; rs13036957 wherein position 41255110 is a G; rs752257 wherein position 22630289 is a G; rs17027230 wherein position 102537848 is a C; rs757863 wherein position 77316032 is an A; rs10740320 wherein position 70840449 is a G; rs4263905 wherein position 145052983 is a T; rs10942784 wherein position 75889806 is an A; rs10809959 wherein position 13497924 is a C; rs10762294 wherein position 70840387 is a C; rs1466471 wherein position 61478245 is a G; rs3744477 wherein position 40183199 is a T; rs10748358 wherein position 421-49850 is a T; rs12119273 wherein position 61655314 is a G; rs10834819 wherein position 25821137 is a G; rs10506228 wherein position 42150219 is a T; rs12995017 wherein position 205046522 is an A; rs945699 wherein position 224400054 is a G; rs1554914 wherein position 150549225 is a T; rs4287603 wherein position 2722492 is a G; rs1027615 wherein position 41998556 is an A; rs666481 wherein position 10010682 is a C; rs1447830 wherein position 74695861 is a C; rs12473579 wherein position 203030073 is a G; rs905080 wherein position 41995195 is a G; rs2205545 wherein position 150677351 is an A; rs3771150 wherein position 102519369 is a C; rs1891592 wherein position 148367576 is an A; rs3749870 wherein position 155646464 is a G; rs12279181 wherein position 25819399 is an A; rs11172457 wherein position 56752884 is a G; rs733281 wherein position 41264461 is a T; rs4819840 wherein position 18096320 is an A; rs4491817 wherein position 18097369 is a G; rs1314625 wherein position 26844530 is a C; rs4516412 wherein position 203029371 is a G; rs879012 wherein position 957788 is a G; rs27628 wherein position 50266128 is a T; rs276915 wherein position 26853979 is an A; rs38271 wherein position 14080271 is a C; rs276916 wherein position 26854159 is a C; rs7772593 wherein position 106451750 is a T; rs7937375 wherein position 21698795 is an A; rs27248 wherein position 50268304 is an A; rs4622670 wherein position 29357853 is a G; and; rs7818421 wherein position 8328291 is a C. The presence of one or more of the tag SNPs listed above detects amyotrophic lateral sclerosis in the human subject, or determining the risk of developing amyotrophic lateral sclerosis in the human subject. The method can include detecting any combination or sub-combination of the tag SNPs. In one example, the method includes detecting all of the tag SNPs.

In one embodiment, the method includes detecting the presence of a tag SNP in the FLJ10986 gene. For example, the method can include detecting the presence of a tag SNP in a nucleic acid encoding

MGISKDPIFV PGVWGPYFSA MVPGFWLNEG GQSVTGKLID
HMVQGHAAFP ELQVKATARC QSIYAYLNSH LDLIKKAQPV
GFLTVDLHVW PDFHGNRSPL ADLTLKGMVT GLKLSQDLDD
LAILYLATVQ AIALGTRFII EAMEAAGHSI STLFLCGGLS
KNPLFVQMHA DITGMPVVLS QEVESVLVGA AVLGACASGD
FASVQEAMAK MSKVGKVVFP RLQDKKYYDK KYQVFLKLVE
HQKEYLAIMN DDL

(SEQ ID NO: 67, see GenBank Accession No. AAQ02454, Jun. 8, 2005, which is herein incorporated by reference).

Thus, the method can include, or can consist of, detecting a haplotype block including rs6690993, wherein position 59416003 is a G and rs6700125, wherein position 59414818 is a T. The method can also include, or can consist of, detecting rs6690993, wherein position 59416003 is a G and/or rs6700125, wherein position 59414818 is a T.

In additional embodiments, the method can include, or can consist of, detecting a tag SNP in the gene encoding anaplastic lymphoma kinase, NADPH oxidase 4 (NOX4), or IQ motif containing GTPase activating protein 2.

Methods are also provided for detecting the genetic predisposition of a subject to bulbar onset ALS. The method can detect early onset ALS in a human subject. The method can also detect the risk of developing bulbar onset ALS in a human subject. The method can also be used to determine if the subject is amenable to treatment with RILUZOLE™.

The methods include detecting the presence of a haplotype block comprising a tag single nucleotide polymorphism (SNP). The method can include detecting at least five, at least ten, or at least fifteen different haplotype blocks, each including a different tag SNP. Detecting the presence of the haplotype block can include detecting a SNP with r² value of greater than about 0.8, about 0.85, about 0.9 or about 0.95 from a tag SNP.

In several embodiments, the tag SNP is rs12695988 wherein position 154604997 is an A; rs4680060 wherein position 154601610 is a T; rs988213 wherein position 42378964 is a G; rs10884751 wherein position 111100812 is an A; rs7806370 wherein position 38461063 is a C; rs6677714 wherein position 236530180 is an A; rs2247691 wherein position 41199732 is a T; rs11233487 wherein position 82529791 is a T; rs17667053 wherein position 70704931 is a C; rs7193888 wherein position 82653630 is a T; rs27628 wherein position 50266127 is a T; rs27248 wherein position 50268303 is an A; rs17741655 wherein position 127147541 is a G; rs4745434 wherein position 75515725 is a T; rs13398914 wherein position 127152871 is an A; rs7740727 wherein position 5654334 is a G; rs11711863 wherein position 185808656 is a C; or rs3944131 wherein position 92386146 is a C. The presence of one or more of the haplotype blocks determines the genetic predisposition to bulbar onset ALS in the human subject. The presence of the one or more haplotype blocks determines the genetic predisposition to bulbar onset ALS in the human subject. The presence of the one or more haplotype blocks determines if the subject can be treated with RILUZOLE™. The method can included detecting the presence of at least five, at least ten, at least eleven, twelve, thirteen, fourteen, fifteen, sixteen, seventeen, or eighteen haplotype bocks, each including a different one of the tag SNPs. The method can included detecting any combination or sub-combination of these haplotype blocks including the tag SNPs. The groups of haplotype blocks can be in any combination, of five, ten, eleven, twelve, thirteen, fourteen, fifteen, sixteen, seventeen, or eighteen haplotype blocks.

The method can also include detecting one of more of the tag SNPs themselves. Thus, the method can include detecting at least five, at least ten, at least eleven, twelve, thirteen, fourteen, fifteen, sixteen, seventeen, or eighteen of the tag SNPs. The method can included detecting any combination or sub-combination of these tag SNPs. The groups of tag SNPs can be in any combination, of five, ten, eleven, twelve, thirteen, fourteen, fifteen, sixteen, seventeen, or eighteen tag SNPs.

With regard to the SNPs, the SNPs are identified by name. The exact sequence of the SNP can be determined from the database of SNPs available at the NCBI website (Entrez SNP, dbSNP build 128). The “position” is the location in the genome of the SNP, referring to the nucleotide position for the p-terminus of the chromosome in the human genome, see the NCBI SNP website, available on the internet.

Methods are also provided for detecting the genetic predisposition of a subject to early onset ALS. The method can detect early onset ALS in a human subject. The method can also detect the risk of developing early onset ALS in a human subject. In this manner, a subject can be identified for treatment with a specific therapeutic agent of interest.

The methods include detecting the presence of a haplotype block comprising a tag single nucleotide polymorphism (SNP). The method can include detecting at least five, at least ten, or at least fifteen different haplotype blocks, each including a different tag SNP. Detecting the presence of the haplotype block can include detecting a SNP with r² value of greater than about 0.8, about 0.85, about 0.9 or about 0.95 from a tag SNP.

In several embodiments, the tag SNP is rs12471471 wherein position 213848557 is an A; rs7569588 wherein position 45331732 is a T; rs12929266 wherein position 49453731 is a T; rs1390762 wherein position 49452674 is an A; rs11096490 wherein position 17949476 is a G; rs4245528 wherein position 106480927 is an A; rs17118549 wherein position 59196347 is a T; rs16983965 wherein position 17951571 is a T; rs10438441 wherein position 90663620 is a T; rs2919708 wherein position 70660625 is a G; rs11089823 wherein position 35833678 is a C; rs38271 wherein position 14080270 is a C; rs838732 wherein position 234103751 is a T; rs2010435 wherein position 82528143 is an A; rs11233487 wherein position 82529791 is an A; rs7171883 wherein position 90664487 is an A; rs2093689 wherein position 94150134 is an A; rs11914132 wherein position 35833586 is a T; rs9558712 wherein position 105646374 is a G; rs3020040 wherein position 70661830 is an A; rs838731 wherein position 234097362 is a C; rs11751085 wherein position 155653676 is a C; rs10224956 wherein position 32969593 is a G; rs3936139 wherein position 2538575 is a C; rs7467398 wherein position 7392207 is a G; rs6772591 wherein position 171997451 is a G; rs13236414 wherein position 32969673 is a G; or rs1943934 wherein position 69938052 is an A.

The presence of one or more of the haplotype blocks determines the genetic predisposition to early onset ALS in the human subject. The presence of the one or more haplotype blocks determines the genetic predisposition to early onset ALS in the human subject. The method can included detecting the presence of at least five, at least ten, at least fifteen, at least 20, 21, 22, 23, 24 or 25 different haplotype bocks, each including a different one of the tag SNPs. The method can included detecting any combination or sub-combination of these tag SNPs. The groups of haplotype blocks can be in any combination, of five, ten, fifteen, 20, 21, 22, 23, 24 or 25 different haplotype blocks.

The method can also include detecting one of more of the tag SNPs themselves. Thus, the method can include detecting at least five, at least ten, at least fifteen, at least 20, 21, 22, 23, 24 or 25 of the different tag SNPs. The method can included detecting any combination or sub-combination of these tag SNPs. The groups of tag SNPs can be in any combination, of five, ten, fifteen, 20, 21, 22, 23, 24 or 25 different tag SNPs.

With regard to the SNPs, the SNPs are identified by name. The exact sequence of the SNP can be determined from the database of SNPs available at the NCBI website (Entrez SNP, dbSNP build 128). The “position” is the location in the genome of the SNP, referring to the nucleotide position for the p-terminus of the chromosome in the human genome, see the NCBI SNP website, available on the internet. Sequence information for each of the tag SNPs listed above is provided in the following table:

### Molecular Methods

Generally, the methods disclosed herein involve an assessment of nucleic acid sequence.

Molecular techniques of use in all of these methods are disclosed below.

Preparation of Nucleic Acids for Analysis: Nucleic acid molecules can be prepared for analysis using any technique known to those skilled in the art. Generally, such techniques result in the production of a nucleic acid molecule sufficiently pure to determine the presence or absence of one or more variations at one or more locations in the nucleic acid molecule. Such techniques are described for example, in Sambrook, et al., Molecular Cloning: A Laboratory Manual (Cold Spring Harbor Laboratory, New York) (1989), and Ausubel, et al., Current Protocols in Molecular Biology (John Wiley and Sons, New York) (1997), incorporated herein by reference.

When the nucleic acid of interest is present in a cell, it can be necessary to first prepare an extract of the cell and then perform further steps, such as differential precipitation, column chromatography, extraction with organic solvents and the like, in order to obtain a sufficiently pure preparation of nucleic acid. Extracts can be prepared using standard techniques in the art, for example, by chemical or mechanical lysis of the cell. Extracts then can be further treated, for example, by filtration and/or centrifugation and/or with chaotropic salts such as guanidinium isothiocyanate or urea or with organic solvents such as phenol and/or HCCl₃ to denature any contaminating and potentially interfering proteins. When chaotropic salts are used, it can be desirable to remove the salts from the nucleic acid-containing sample. This can be accomplished using standard techniques in the art such as precipitation, filtration, size exclusion chromatography and the like.

In some instances, messenger RNA can be extracted from cells. Techniques and material for this purpose are known to those skilled in the art and can involve the use of oligo dT attached to a solid support such as a bead or plastic surface. In some embodiments, the mRNA can be reversed transcribed into cDNA using, for example, a reverse transcriptase enzyme. Suitable enzymes are commercially available from, for example, Invitrogen, Carlsbad Calif. Optionally, cDNA prepared from mRNA can also be amplified.

Amplification of nucleic acid molecules: Optionally, the nucleic acid samples obtained from the subject are amplified prior to detection. Target nucleic acids are amplified to obtain amplification products, including sequences from a haplotype block including a tag SNP, can be amplified from the sample prior to detection. Typically, DNA sequences are amplified, although in some instances RNA sequences can be amplified or converted into cDNA, such as by using RT PCR.

Any nucleic acid amplification method can be used. An example of in vitro amplification is the polymerase chain reaction (PCR), in which a biological sample obtained from a subject is contacted with a pair of oligonucleotide primers, under conditions that allow for hybridization of the primers to a nucleic acid molecule in the sample. The primers are extended under suitable conditions, dissociated from the template, and then re-annealed, extended, and dissociated to amplify the number of copies of the nucleic acid molecule. Other examples of in vitro amplification techniques include quantitative real-time PCR, strand displacement amplification (see U.S. Pat. No. 5,744,311); transcription-free isothermal amplification (see U.S. Pat. No. 6,033,881); repair chain reaction amplification (see PCT Publication NO. WO 90/01069); ligase chain reaction amplification (see EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134).

In specific examples, the target sequences to be amplified from the subject include one or different haplotype blocks including a tag SNP, or a nucleotide sequence of interest including the tag SNP. In certain embodiments, target sequences containing one or more of SEQ ID NOs: 1-53, or a subset thereof, are amplified. In an embodiment, a single marker with exceptionally high predictive value is amplified, or a nucleic acid encoding FLJ10986 is amplified.

A pair of primers can be utilized in the amplification reaction. One or both of the primers can be labeled, for example with a detectable radiolabel, fluorophore, or biotin molecule. The pair of primers includes an upstream primer (which binds 5′ to the downstream primer) and a downstream primer (which binds 3′ to the upstream primer). The pair of primers used in the amplification reactions are selective primers which permit amplification of a size related marker locus. Primers can be selected to amplify a haplotype block including a tag SNP, or a nucleic acid including a tag SNP. Numerous primers can be designed by those of skill in the art simply by determining the sequence of the desired target region, for example, using well known computer assisted algorithms that select primers within desired parameters suitable for annealing and amplification.

If desired, an additional pair of primers can be included in the amplification reaction as an internal control. For example, these primers can be used to amplify a “housekeeping” nucleic acid molecule, and serve to provide confirmation of appropriate amplification. In another example, a target nucleic acid molecule including primer hybridization sites can be constructed and included in the amplification reactor. One of skill in the art will readily be able to identify primer pairs to serve as internal control primers.

Primer Design Strategy: Increased use of polymerase chain reaction (PCR) methods has stimulated the development of many programs to aid in the design or selection of oligonucleotides used as primers for PCR. Four examples of such programs that are freely available via the Internet are: PRIMER™ by Mark Daly and Steve Lincoln of the Whitehead Institute (UNIX, VMS, DOS, and Macintosh), Oligonucleotide Selection Program by Phil Green and LaDeana Hiller of Washington University in St. Louis (UNIX, VMS, DOS, and Macintosh), PGEN™ by Yoshi (DOS only), and Amplify by Bill Engels of the University of Wisconsin (Macintosh only). Generally these programs help in the design of PCR primers by searching for bits of known repeated-sequence elements and then optimizing the Tₘ by analyzing the length and GC content of a putative primer. Commercial software is also available and primer selection procedures are rapidly being included in most general sequence analysis packages.

Designing oligonucleotides for use as either sequencing or PCR primers to detect requires selection of an appropriate sequence that specifically recognizes the target, and then testing the sequence to eliminate the possibility that the oligonucleotide will have a stable secondary structure. Inverted repeats in the sequence can be identified using a repeat-identification or RNA-folding programs. If a possible stem structure is observed, the sequence of the primer can be shifted a few nucleotides in either direction to minimize the predicted secondary structure. When the amplified sequence is intended for subsequence cloning, the sequence of the oligonucleotide can also be compared with the sequences of both strands of the appropriate vector and insert DNA. A sequencing primer only has a single match to the target DNA. It is also advisable to exclude primers that have only a single mismatch with an undesired target DNA sequence. For PCR primers used to amplify genomic DNA, the primer sequence can be compared to the sequences in the GENBANK™ database to determine if any significant matches occur. If the oligonucleotide sequence is present in any known DNA sequence or, more importantly, in any known repetitive elements, the primer sequence should be changed.

Detection of alleles: The nucleic acids obtained from the sample can be genotyped to identify the particular allele present for a marker locus. A sample of sufficient quantity to permit direct detection of marker alleles from the sample can be obtained from the subject Alternatively, a smaller sample is obtained from the subject and the nucleic acids are amplified prior to detection. Any target nucleic that is informative for a chromosome haplotype can be detected. Generally, the target nucleic acid corresponds to a tag SNP described above, or an SNP described above. Any method of detecting a nucleic acid molecule can be used, such as hybridization and/or sequencing assays.

Hybridization is the binding of complementary strands of DNA, DNA/RNA, or RNA. Hybridization can occur when primers or probes bind to target sequences such as target sequences within genomic DNA. Probes and primers that are useful generally include nucleic acid sequences that hybridize (for example under high stringency conditions) with a nucleic acid sequence including the tag SNP of interest, but do not hybridize to a wild-type allele, or that hybridize to the wild-type allele, but do not hybridize to the tag SNP. Physical methods of detecting hybridization or binding of complementary strands of nucleic acid molecules, include but are not limited to, such methods as DNase I or chemical footprinting, gel shift and affinity cleavage assays, Southern and Northern blotting, dot blotting and light absorption detection procedures. The binding between a nucleic acid primer or probe and its target nucleic acid is frequently characterized by the temperature (Tₘ) at which 50% of the nucleic acid probe is melted from its target. A higher (Tₘ) means a stronger or more stable complex relative to a complex with a lower (Tm).

Generally, complementary nucleic acids form a stable duplex or triplex when the strands bind, (hybridize), to each other by forming Watson-Crick, Hoogsteen or reverse Hoogsteen base pairs. Stable binding occurs when an oligonucleotide molecule remains detectably bound to a target nucleic acid sequence under the required conditions.

Complementarity is the degree to which bases in one nucleic acid strand base pair with the bases in a second nucleic acid strand. Complementarity is conveniently described by percentage, that is, the proportion of nucleotides that form base pairs between two strands or within a specific region or domain of two strands. For example, if 10 nucleotides of a 15-nucleotide oligonucleotide form base pairs with a targeted region of a DNA molecule, that oligonucleotide is said to have 66.67% complementarity to the region of DNA targeted.

In the present disclosure, “sufficient complementarity” means that a sufficient number of base pairs exist between an oligonucleotide molecule and a target nucleic acid sequence (such as a tag SNP) to achieve detectable and specific binding. When expressed or measured by percentage of base pairs formed, the percentage complementarity that fulfills this goal can range from as little as about 50% complementarity to full (100%) complementary. In general, sufficient complementarity is at least about 50%, for example at least about 75% complementarity, at least about 90% complementarity, at least about 95% complementarity, at least about 98% complementarity, or even at least about 100% complementarity. The qualitative and quantitative considerations involved in establishing binding conditions that allow one skilled in the art to design appropriate oligonucleotides for use under the desired conditions is provided by Beltz et al. Methods Enzymol 100:266-285, 1983, and by Sambrook et al. (ed.), Molecular Cloning: A Laboratory Manual, 2nd ed., vol. 1-3, Cold Spring Harbor Laboratory Press, Cold Spring Harbor, N.Y., 1989.

Hybridization conditions resulting in particular degrees of stringency will vary depending upon the nature of the hybridization method and the composition and length of the hybridizing nucleic acid sequences. Generally, the temperature of hybridization and the ionic strength (such as the Na+ concentration) of the hybridization buffer will determine the stringency of hybridization. Calculations regarding hybridization conditions for attaining particular degrees of stringency are discussed in Sambrook et al., (1989) Molecular Cloning: a laboratory manual, second edition, Cold Spring Harbor Laboratory, Plainview, N.Y. (chapters 9 and 11). The following is an exemplary set of hybridization conditions and is not limiting:

Very High Stringency (Detects Sequences that Share at Least 90% Complementarity)

| | |
|---|---|
| Hybridization: | 5x SSC at 65° C. for 16 hours |
| Wash twice: | 2x SSC at room temperature (RT) for 15 minutes each |
| Wash twice: | 0.5x SSC at 65° C. for 20 minutes each |

High Stringency (Detects Sequences that Share at Least 80% Complementarity)

| | |
|---|---|
| Hybridization: | 5x-6x SSC at 65° C.-70° C. for 16-20 hours |
| Wash twice: | 2x SSC at RT for 5-20 minutes each |
| Wash twice: | 1x SSC at 55° C.-70° C. for 30 minutes each |

Low Stringency (Detects Sequences that Share at Least 50% Complementarity)

| | |
|---|---|
| Hybridization: | 6x SSC at RT to 55° C. for 16-20 hours |
| Wash at least twice: | 2x-3x SSC at RT to 55° C. for 20-30 minutes each. |

Methods for labeling nucleic acid molecules so they can be detected are well known. Examples of such labels include non-radiolabels and radiolabels. Non-radiolabels include, but are not limited to an enzyme, chemiluminescent compound, fluorescent compound (such as FITC, Cy3, and Cy5), metal complex, hapten, enzyme, colorimetric agent, a dye, or combinations thereof. Radiolabels include, but are not limited to, ¹²⁵I and ³⁵S. For example, radioactive and fluorescent labeling methods, as well as other methods known in the art, are suitable for use with the present disclosure. In one example, primers used to amplify the subject's nucleic acids are labeled (such as with biotin, a radiolabel, or a fluorophore). In another example, amplified target nucleic acid samples are end-labeled to form labeled amplified material. For example, amplified nucleic acid molecules can be labeled by including labeled nucleotides in the amplification reactions.

Nucleic acid molecules corresponding to one or tag SNPs or haplotype blocks including the tag SNPs can also be detected by hybridization procedures using a labeled nucleic acid probe, such as a probe that detects only one alternative allele at a marker locus. Most commonly, the target nucleic acid (or amplified target nucleic acid) is separated based on size or charge and transferred to a solid support. The solid support (such as membrane made of nylon or nitrocellulose) is contacted with a labeled nucleic acid probe, which hybridizes to it complementary target under suitable hybridization conditions to form a hybridization complex. Hybridization conditions for a given combination of array and target material can be optimized routinely in an empirical manner close to the Tₘ of the expected duplexes, thereby maximizing the discriminating power of the method. For example, the hybridization conditions can be selected to permit discrimination between matched and mismatched oligonucleotides. Hybridization conditions can be chosen to correspond to those known to be suitable in standard procedures for hybridization to filters (and optionally for hybridization to arrays). In particular, temperature is controlled to substantially eliminate formation of duplexes between sequences other than an exactly complementary allele of the selected marker. A variety of known hybridization solvents can be employed, the choice being dependent on considerations known to one of skill in the art (see U.S. Pat. No. 5,981,185).

Once the target nucleic acid molecules have been hybridized with the labeled probes, the presence of the hybridization complex can be analyzed, for example by detecting the complexes.

Methods for detecting hybridized nucleic acid complexes are well known in the art. In one example, detection includes detecting one or more labels present on the oligonucleotides, the target (e.g., amplified) sequences, or both. Detection can include treating the hybridized complex with a buffer and/or a conjugating solution to effect conjugation or coupling of the hybridized complex with the detection label, and treating the conjugated, hybridized complex with a detection reagent. In one example, the conjugating solution includes streptavidin alkaline phosphatase, avidin alkaline phosphatase, or horseradish peroxidase. Specific, non-limiting examples of conjugating solutions include streptavidin alkaline phosphatase, avidin alkaline phosphatase, or horseradish peroxidase. The conjugated, hybridized complex can be treated with a detection reagent. In one example, the detection reagent includes enzyme-labeled fluorescence reagents or calorimetric reagents. In one specific non-limiting example, the detection reagent is enzyme-labeled fluorescence reagent (ELF) from Molecular Probes, Inc. (Eugene, Oreg.). The hybridized complex can then be placed on a detection device, such as an ultraviolet (UV) transilluminator (manufactured by UVP, Inc. of Upland, Calif.). The signal is developed and the increased signal intensity can be recorded with a recording device, such as a charge coupled device (CCD) camera (manufactured by Photometrics, Inc. of Tucson, Ariz.). In particular examples, these steps are not performed when radiolabels are used. In particular examples, the method further includes quantification, for instance by determining the amount of hybridization.

Allele Specific PCR: Allele-specific PCR differentiates between target regions differing in the presence of absence of a variation or polymorphism. PCR amplification primers are chosen based upon their complementarity to the target sequence, such as nucleic acid sequence in a haplotype block including a tag SNP, a specified region of an allele including a tag SNP, or to the tag SNP itself The primers bind only to certain alleles of the target sequence. This method is described by Gibbs, Nucleic Acid Res. 17:12427 2448, 1989, herein incorporated by reference.

Allele Specific Oligonucleotide Screening Methods: Further screening methods employ the allele-specific oligonucleotide (ASO) screening methods (e.g. see Saiki et al., Nature 324:163-166, 1986). Oligonucleotides with one or more base pair mismatches are generated for any particular allele or haplotype block. ASO screening methods detect mismatches between one allele (or haplotype block) in the target genomic or PCR amplified DNA and the other allele (or haplotype block), showing decreased binding of the oligonucleotide relative to the second allele (i.e. the other allele) oligonucleotide. Oligonucleotide probes can be designed that under low stringency will bind to both polymorphic forms of the allele, but which at high stringency, only bind to the allele to which they correspond. Alternatively, stringency conditions can be devised in which an essentially binary response is obtained, i.e., an ASO corresponding to a variant form of the target gene will hybridize to that allele (haplotype block), and not to the wildtype allele (haplotype block).

Ligase Mediated Allele Detection Method: Ligase can also be used to detect point mutations, such as the tag SNPs disclosed herein, in a ligation amplification reaction (e.g. as described in Wu et al., Genomics 4:560-569, 1989). The ligation amplification reaction (LAR) utilizes amplification of specific DNA sequence using sequential rounds of template dependent ligation (e.g. as described in Wu, supra, and Barany, Proc. Nat. Acad. Sci. 88:189-193, 1990).

Denaturing Gradient Gel Electrophoresis: Amplification products generated using the polymerase chain reaction can be analyzed by the use of denaturing gradient gel electrophoresis. Different alleles (haplotype blocks) can be identified based on the different sequence-dependent melting properties and electrophoretic migration of DNA in solution. DNA molecules melt in segments, termed melting domains, under conditions of increased temperature or denaturation. Each melting domain melts cooperatively at a distinct, base-specific melting temperature (T_{M}). Melting domains are at least 20 base pairs in length, and can be up to several hundred base pairs in length.

Differentiation between alleles (haplotype blocks) based on sequence specific melting domain differences can be assessed using polyacrylamide gel electrophoresis, as described in Chapter 7 of Erlich, ed., PCR Technology, Principles and Applications for DNA Amplification, W.H. Freeman and Co., New York (1992).

Generally, a target region to be analyzed by denaturing gradient gel electrophoresis is amplified using PCR primers flanking the target region. The amplified PCR product is applied to a polyacrylamide gel with a linear denaturing gradient as described in Myers et al., Meth. Enzymol. 155:501-527, 1986, and Myers et al., in Genomic Analysis, A Practical Approach, K. Davies Ed. IRL Press Limited, Oxford, pp. 95 139, 1988. The electrophoresis system is maintained at a temperature slightly below the Tm of the melting domains of the target sequences.

In an alternative method of denaturing gradient gel electrophoresis, the target sequences can be initially attached to a stretch of GC nucleotides, termed a GC clamp, as described in Chapter 7 of Erlich, supra. In one example, at least 80% of the nucleotides in the GC clamp are either guanine or cytosine. In another example, the GC clamp is at least 30 bases long. This method is particularly suited to target sequences with high Tₘ's.

Generally, the target region is amplified by polymerase chain reaction. One of the oligonucleotide PCR primers carries at its 5′ end, the GC clamp region, at least 30 bases of the GC rich sequence, which is incorporated into the 5′ end of the target region during amplification. The resulting amplified target region is run on an electrophoresis gel under denaturing gradient conditions. DNA fragments differing by a single base change will migrate through the gel to different positions, which can be visualized by ethidium bromide staining.

Temperature Gradient Gel Electrophoresis: Temperature gradient gel electrophoresis (TGGE) is based on the same underlying principles as denaturing gradient gel electrophoresis, except the denaturing gradient is produced by differences in temperature instead of differences in the concentration of a chemical denaturant. Standard TGGE utilizes an electrophoresis apparatus with a temperature gradient running along the electrophoresis path. As samples migrate through a gel with a uniform concentration of a chemical denaturant, they encounter increasing temperatures. An alternative method of TGGE, temporal temperature gradient gel electrophoresis (TTGE or tTGGE) uses a steadily increasing temperature of the entire electrophoresis gel to achieve the same result. As the samples migrate through the gel the temperature of the entire gel increases, leading the samples to encounter increasing temperature as they migrate through the gel. Preparation of samples, including PCR amplification with incorporation of a GC clamp, and visualization of products are the same as for denaturing gradient gel electrophoresis.

Single-Strand Conformation Polymorphism Analysis: Target sequences, such as alleles or haplotype blocks can be differentiated using single-strand conformation polymorphism analysis, which identifies base differences by alteration in electrophoretic migration of single stranded PCR products, for example as described in Orita et al. Proc. Nat. Acad. Sci. 85:2766-2770, 1989. Amplified PCR products can be generated as described above, and heated or otherwise denatured, to form single stranded amplification products. Single-stranded nucleic acids can refold or form secondary structures which are partially dependent on the base sequence. Thus, electrophoretic mobility of single-stranded amplification products can detect base-sequence difference between alleles or haplotype blocks.

Chemical or Enzymatic Cleavage of Mismatches: Differences between target sequences, such as alleles or haplotype blocks, can also be detected by differential chemical cleavage of mismatched base pairs, for example as described in Grompe et al., Am. J. Hum. Genet. 48:212-222, 1991. In another method, differences between target sequences, such as alleles or haplotype blocks, can be detected by enzymatic cleavage of mismatched base pairs, as described in Nelson et al., Nature Genetics 4:11-18, 1993. Briefly, genetic material from an animal and an affected family member can be used to generate mismatch free heterohybrid DNA duplexes. As used herein, “heterohybrid” means a DNA duplex strand comprising one strand of DNA from one animal, and a second DNA strand from another animal, usually an animal differing in the phenotype for the trait of interest. Positive selection for heterohybrids free of mismatches allows determination of small insertions, deletions or other polymorphisms

Non-gel Systems: Other possible techniques include non-gel systems such as TaqMan™ (Perkin Elmer). In this system oligonucleotide PCR primers are designed that flank the mutation in question and allow PCR amplification of the region. A third oligonucleotide probe is then designed to hybridize to the region containing the base subject to change between different alleles of the gene. This probe is labeled with fluorescent dyes at both the 5′ and 3′ ends. These dyes are chosen such that while in this proximity to each other the fluorescence of one of them is quenched by the other and cannot be detected. Extension by Taq DNA polymerase from the PCR primer positioned 5′ on the template relative to the probe leads to the cleavage of the dye attached to the 5′ end of the annealed probe through the 5′ nuclease activity of the Taq DNA polymerase. This removes the quenching effect allowing detection of the fluorescence from the dye at the 3′ end of the probe. The discrimination between different DNA sequences arises through the fact that if the hybridization of the probe to the template molecule is not complete (there is a mismatch of some form) the cleavage of the dye does not take place. Thus only if the nucleotide sequence of the oligonucleotide probe is completely complimentary to the template molecule to which it is bound will quenching be removed. A reaction mix can contain two different probe sequences each designed against different alleles that might be present thus allowing the detection of both alleles in one reaction.

Non-PCR Based Allele detection: The identification of a DNA sequence can be made without an amplification step, based on polymorphisms including restriction fragment length polymorphisms in a subject and a control, such as a family member. Hybridization probes are generally oligonucleotides which bind through complementary base pairing to all or part of a target nucleic acid. Probes typically bind target sequences lacking complete complementarity with the probe sequence depending on the stringency of the hybridization conditions. The probes can be labeled directly or indirectly, such that by assaying for the presence or absence of the probe, one can detect the presence or absence of the target sequence. Direct labeling methods include radioisotope labeling, such as with ³²P or ³⁵S. Indirect labeling methods include fluorescent tags, biotin complexes which can be bound to avidin or streptavidin, or peptide or protein tags. Visual detection methods include photoluminescents, Texas red, rhodamine and its derivatives, red leuco dye and 3,3′,5,5′-tetramethylbenzidine (TMB), fluorescein, and its derivatives, dansyl, umbelliferone and the like or with horse radish peroxidase, alkaline phosphatase and the like.

Hybridization probes include any nucleotide sequence capable of hybridizing to a nucleic acid sequence wherein a polymorphism is present that is associated with ALS, such as a tag SNP, and thus defining a genetic marker, including a restriction fragment length polymorphism, a hypervariable region, repetitive element, or a variable number tandem repeat. Hybridization probes can be any gene or a suitable analog. Further suitable hybridization probes include exon fragments or portions of cDNAs or genes known to map to the relevant region of the chromosome.

Exemplary tandem repeat hybridization probes for use in the methods disclosed are those that recognize a small number of fragments at a specific locus at high stringency hybridization conditions, or that recognize a larger number of fragments at that locus when the stringency conditions are lowered.

Arrays for detecting nucleic acid: In particular examples involving genotyping of multiple marker loci, the methods can be performed using an array that includes a plurality of markers. Such arrays can include nucleic acid molecules. In one example, the array includes nucleic acid oligonucleotide probes that can hybridize to one or more alleles.

Arrays can be used to detect the presence of amplified sequences including one or more tag SNPs of interest using specific oligonucleotide probes. In one example, a set of oligonucleotide probes is attached to the surface of a solid support for use in detection of marker alleles that define haplotypes that determine a genetic predisposition to ALS, bulbar onset ALS or early onset ALS. Additionally, if an internal control nucleic acid sequence was amplified in the amplification reaction (see above), an oligonucleotide probe can be included to detect the presence of this amplified nucleic acid molecule. The oligonucleotide probes bound to the array can specifically bind sequences amplified in the amplification reaction (such as under high stringency conditions).

The methods and apparatus in accordance with the present disclosure takes advantage of the fact that under appropriate conditions oligonucleotides form base-paired duplexes with nucleic acid molecules that have a complementary base sequence. The stability of the duplex is dependent on a number of factors, including the length of the oligonucleotides, the base composition, and the composition of the solution in which hybridization is effected. The effects of base composition on duplex stability can be reduced by carrying out the hybridization in particular solutions, for example in the presence of high concentrations of tertiary or quaternary amines.

The thermal stability of the duplex is also dependent on the degree of sequence similarity between the sequences. By carrying out the hybridization at temperatures close to the anticipated Tₘ's of the type of duplexes expected to be formed between the target sequences and the oligonucleotides bound to the array, the rate of formation of mis-matched duplexes can be substantially reduced.

The length of each oligonucleotide sequence employed in the array can be selected to optimize binding to a specific allele of a marker locus associated with ALS. An optimum length for use with a particular marker nucleic acid sequence under specific screening conditions can be determined empirically. Thus, the length for each individual element of the set of oligonucleotide sequences included in the array can be optimized for screening. In one example, oligonucleotide probes are from about 20 to about 35 nucleotides in length or about 25 to about 40 nucleotides in length.

The oligonucleotide probe sequences forming the array can be directly linked to the support, for example via the 5′—or 3′-end of the probe. In one example, the oligonucleotides are bound to the solid support by the 5′ end. However, one of skill in the art can determine whether the use of the 3′ end or the 5′ end of the oligonucleotide is suitable for bonding to the solid support. In general, the internal complementarity of an oligonucleotide probe in the region of the 3′ end and the 5′ end determines binding to the support. Alternatively, the oligonucleotide probes can be attached to the support by sequences such as oligonucleotides or other molecules that serve as spacers or linkers to the solid support.

In particular examples, the array is a microarray formed from glass (silicon dioxide). Suitable silicon dioxide types for the solid support include, but are not limited to: aluminosilicate, borosilicate, silica, soda lime, zinc titania and fused silica (for example see Schena, Micraoarray Analysis. John Wiley & Sons, Inc, Hoboken, N.J., 2003). The attachment of nucleic acids to the surface of the glass can be achieved by methods known in the art, for example by surface treatments that form from an organic polymer. Particular examples include, but are not limited to: polypropylene, polyethylene, polybutylene, polyisobutylene, polybutadiene, polyisoprene, polyvinylpyrrolidine, polytetrafluroethylene, polyvinylidene difluroide, polyfluoroethylene-propylene, polyethylenevinyl alcohol, polymethylpentene, polycholorotrifluoroethylene, polysulformes, hydroxylated biaxially oriented polypropylene, aminated biaxially oriented polypropylene, thiolated biaxially oriented polypropylene, etyleneacrylic acid, thylene methacrylic acid, and blends of copolymers thereof (see U.S. Pat. No. 5,985,567), organosilane compounds that provide chemically active amine or aldehyde groups, epoxy or polylysine treatment of the microarray. Another example of a solid support surface is polypropylene.

In general, suitable characteristics of the material that can be used to form the solid support surface include: being amenable to surface activation such that upon activation, the surface of the support is capable of covalently attaching a biomolecule such as an oligonucleotide thereto; amenability to “in situ” synthesis of biomolecules; being chemically inert such that at the areas on the support not occupied by the oligonucleotides are not amenable to non-specific binding, or when non-specific binding occurs, such materials can be readily removed from the surface without removing the oligonucleotides.

In one example, the surface treatment is amine-containing silane derivatives. Attachment of nucleic acids to an amine surface occurs via interactions between negatively charged phosphate groups on the DNA backbone and positively charged amino groups (Schena, Micraoarray Analysis. John Wiley & Sons, Inc, Hoboken, N.J., 2003). In another example, reactive aldehyde groups are used as surface treatment. Attachment to the aldehyde surface is achieved by the addition of 5′-amine group or amino linker to the DNA of interest. Binding occurs when the nonbonding electron pair on the amine linker acts as a nucleophile that attacks the electropositive carbon atom of the aldehyde group.

A wide variety of array formats can be employed in accordance with the present disclosure. One example includes a linear array of oligonucleotide bands, generally referred to in the art as a dipstick. Another suitable format includes a two-dimensional pattern of discrete cells (such as 4096 squares in a 64 by 64 array). As is appreciated by those skilled in the art, other array formats including, but not limited to slot (rectangular) and circular arrays are equally suitable for use (see U.S. Pat. No. 5,981,185). In one example, the array is formed on a polymer medium, which is a thread, membrane or film. An example of an organic polymer medium is a polypropylene sheet having a thickness on the order of about 1 mil. (0.001 inch) to about 20 mil., although the thickness of the film is not critical and can be varied over a fairly broad range. Biaxially oriented polypropylene (BOPP) films are also suitable in this regard; in addition to their durability, BOPP films exhibit a low background fluorescence. In a particular example, the array is a solid phase, Allele-Specific Oligonucleotides (ASO) based nucleic acid array.

The array formats of the present disclosure can be included in a variety of different types of formats. A “format” includes any format to which the solid support can be affixed, such as microtiter plates, test tubes, inorganic sheets, dipsticks, and the like. For example, when the solid support is a polypropylene thread, one or more polypropylene threads can be affixed to a plastic dipstick-type device; polypropylene membranes can be affixed to glass slides. The particular format is, in and of itself, unimportant. All that is necessary is that the solid support can be affixed thereto without affecting the functional behavior of the solid support or any biopolymer absorbed thereon, and that the format (such as the dipstick or slide) is stable to any materials into which the device is introduced (such as clinical samples and hybridization solutions).

The arrays of the present disclosure can be prepared by a variety of approaches. In one example, oligonucleotide or protein sequences are synthesized separately and then attached to a solid support (see U.S. Pat. No. 6,013,789). In another example, sequences are synthesized directly onto the support to provide the desired array (see U.S. Pat. No. 5,554,501). Suitable methods for covalently coupling oligonucleotides and proteins to a solid support and for directly synthesizing the oligonucleotides or proteins onto the support are known to those working in the field; a summary of suitable methods can be found in Matson et al., Anal. Biochem. 217:306-10, 1994. In one example, the oligonucleotides are synthesized onto the support using conventional chemical techniques for preparing oligonucleotides on solid supports (such as see PCT Publication No. WO 85/01051 and PCT Publication No. WO 89/10977, or U.S. Pat. No. 5,554,501).

A suitable array can be produced using automated means to synthesize oligonucleotides in the cells of the array by laying down the precursors for the four bases in a predetermined pattern. Briefly, a multiple-channel automated chemical delivery system is employed to create oligonucleotide probe populations in parallel rows (corresponding in number to the number of channels in the delivery system) across the substrate. Following completion of oligonucleotide synthesis in a first direction, the substrate can then be rotated by 90° to permit synthesis to proceed within a second)(2° set of rows that are now perpendicular to the first set. This process creates a multiple-channel array whose intersection generates a plurality of discrete cells.

In particular examples, the oligonucleotide probes on the array include one or more labels, which permit detection of oligonucleotide probe:target sequence hybridization complexes.

### Kits

The present disclosure provides for kits that can be used to detect a genetic predisposition to ALS, bulbar onset ALS and early onset ALS. The disclosed kits can include a binding molecule, such as an oligonucleotide probe that selectively hybridizes to an allele of a haplotype block including a tag SNP. In one example, the kit includes the isolated oligonucleotide probes that bind to one or more of the nucleic acid sequences set forth as SEQ ID NOs: 1-53, or probes for five, ten, twenty, thirty, forty of fifty of the nucleic acid sequences set forth of SEQ ID NOs: 1-52, wherein these sequences include the tag SNP associated with ALS.

Alternatively or additionally, the kits can include one or more isolated primers or primer pairs for amplifying a target nucleic acid, such as one or more haplotype blocks including a tag SNP. For example, the kit can include primers for amplifying for five, ten, twenty, thirty, forty of fifty haplotype blocks including a tag SNP, such as the nucleic acid sequences set forth as SEQ ID NOs: 1-53, wherein the sequence includes the tag SNP associated with ALS.

The kit can further include one or more of a buffer solution, a conjugating solution for developing the signal of interest, or a detection reagent for detecting the signal of interest, each in separate packaging, such as a container. In another example, the kit includes a plurality of size-associated marker target nucleic acid sequences for hybridization with a detection array. The target nucleic acid sequences can include oligonucleotides such as DNA, RNA, and peptide-nucleic acid, or can include PCR fragments. The kit can also include instructions in a tangible form, such as written instructions or in a computer-readable format.

### Screening Compounds for an Ability to Modulate ALS-Associated Genes

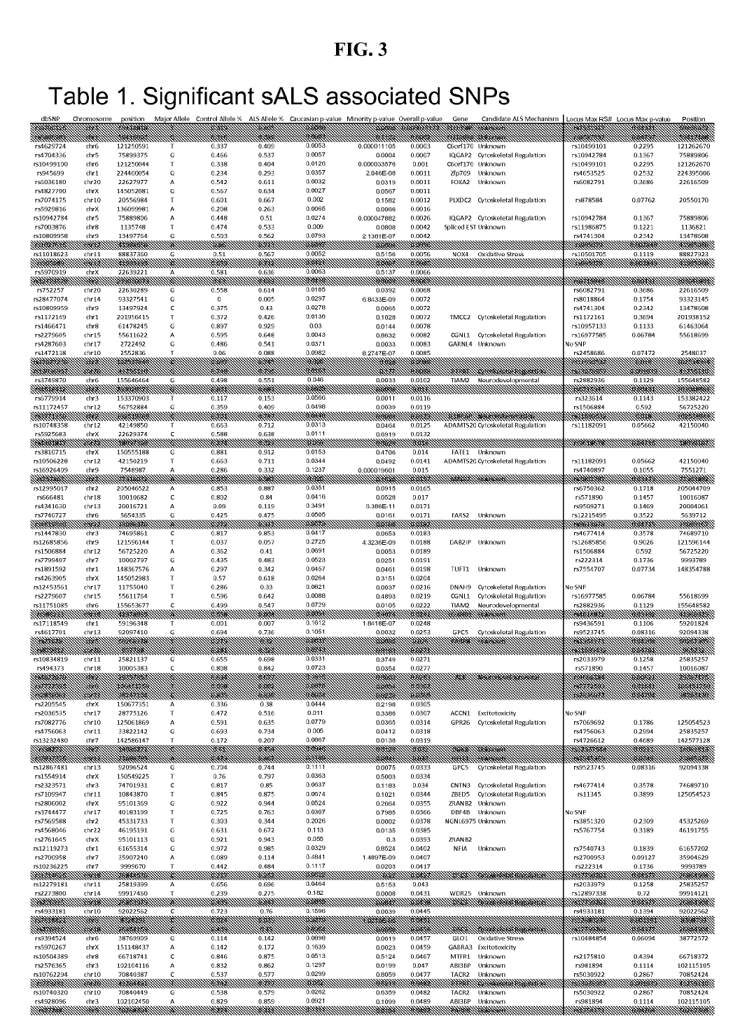

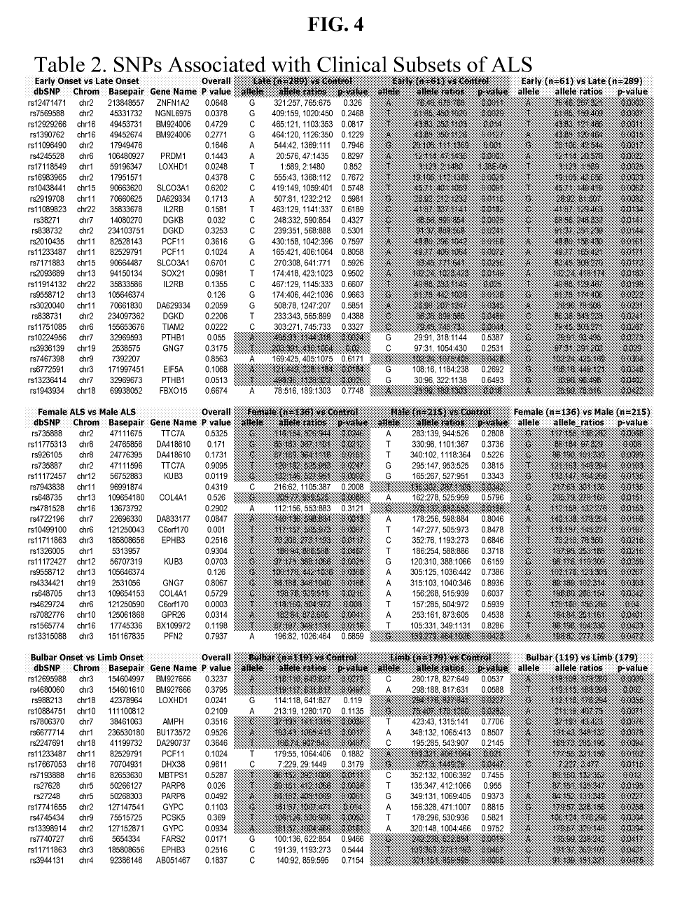

In some embodiments, an agent of compound or combination of agents or compounds are screened to determine if such an agent(s) affects activity least one gene associated with each of the following mechanisms: cytoskeleton or neuronal cell adhesion, oxidative stress, calcium homeostasis, neuroinflammation, glutamate excitotoxicity or neurodevelopment. In one embodiment, an agent(s) is screened to determine if at least one gene activity in from Table 1 or Table 2 (see FIGS. 3 and 4) is modulated. In various embodiments, such agents include but are not limited to nucleic acid molecules, RNAi, antisense, aptamers, small inorganic molecules, antibodies, proteins, peptides or peptide-nucleic acids (PNA).

In one embodiment a bioactive agent or combination of bioactive agent(s) affects the activity of at least one gene. The term “activity” in this context means gene expression levels, protein expression levels or protein biochemical function. For example, a bioactive agent can modulate mRNA levels up or down, modulate protein levels up or down or reduce or enhance protein function (e.g., enzymatic activity). Furthermore, such screening processes can be carried out in vivo (e.g., in an animal subject) or in vitro (e.g., in cell culture).

Assays for variant gene expression can involve direct assays of nucleic acid levels (e.g., mRNA levels), expressed protein levels, or of collateral compounds involved in the signal pathway. Further, the expression of genes that are up- or down-regulated in response to the signal pathway can also be assayed. In this embodiment, the regulatory regions of these genes can be operably linked to a reporter gene such as luciferase.

Modulators of variant gene expression can be identified in a method wherein, for example, a cell is contacted with a candidate compound/agent and the expression of mRNA determined The level of expression of mRNA in the presence of the candidate compound is compared to the level of expression of mRNA in the absence of the candidate compound. The candidate compound can then be identified as a modulator of variant gene expression based on this comparison and be used to treat a disorder such as ALS disease that is characterized by abnormal gene expression due to one or more SNPs of the present invention. When expression of mRNA is statistically significantly greater in the presence of the candidate compound than in its absence, the candidate compound is identified as a stimulator of nucleic acid expression. When nucleic acid expression is statistically significantly less in the presence of the candidate compound than in its absence, the candidate compound is identified as an inhibitor of nucleic acid expression.

By “modulate” it is meant that gene activity is “decreased” or “increased”, and it is intended within the context of this invention that the assessed parameter is between 5% and 90% of the parameter value with a wild-type environment or control environment (e.g., no bioactive agent). In some embodiments, said assessed parameter is between 25% and 75% of the parameter value with a wild-type or control environment. In some embodiments, the assessed parameter is between about 5% and 20%, about 10% and 30%, about 20% and 40%, about 30% and 50%, about 40% and 60%, about 50% and 70%, about 60% and 80%, or about 70% and 90%. In one embodiment, the parameter is about 10%, 20%, 30%, 40%, 50%, 60%, 70%, 80% or 90%. By “abolish”, it is intended within the context of this invention that the assessed parameter is less than 10%, less than 5% or less than 1% of the parameter value with a wild-type or control environment.

The disclosure is illustrated by the following non-limiting Examples.

### EXAMPLES

### Example 1

### Methods

Sample Acquisition For these studies 1,251 DNA samples from individuals with a diagnosis of laboratory—supported probable, probable, or definite ALS using the El Escorial diagnostic criteria (Brooks et al., Amyotroph Lateral Scler Other Motor Neuron Disord. 2000; 1:293-9). Individuals were recruited and enrolled from all participating clinical sites on this study. In addition, 231 ALS DNA samples we obtained from a depository. Samples obtained from the depository were cross-referenced to the prospectively collected patient group and three duplicates were removed. All clinical information for every enrolled subject was entered in an anonymous, coded format and tracked in a fully HIPAA compliant online database 1,152 ALS samples were of sufficient quality to be genotyped (as described below). This total ALS series consisted of 824 Caucasians, 87 Hispanics, 35 African Americans, 8 Asians, 3 American Indians, 3 Pacific Islanders, and 192 unknown ethnicities. There were 692 males and 460 females with a mean age of 59 and mean ALS Functional Rating Scale-Revised (ALSFRS-R) score of 30.37. Carefully matched Caucasian sALS cases were withheld and compared to a population of 700 neurologically cleared control individuals in the training series. The 750 neurologically normal, aged replication series 1 controls were purchased from the Rutgers University Cell and DNA Repository (RUCDR).

Whole Genome Association: Genomic DNA was isolated using the Puregene DNA isolation kit (Gentra Systems, Inc, Minneapolis, Minn.). Prior to quantitation, all DNA samples were checked for quality using 2% agarose gel electrophoresis, and degraded samples (as evidenced by characteristic smearing of DNA to low molecular weight species) were excluded from the high-density whole-genome SNP genotyping assay. Individual genomic DNA concentrations of each subject were determined in quintriplicate with the Quant-iT PicoGreen dsDNA Assay Kit (Invitrogen, Carlsbad, Calif.) according to the manufacturer's instructions. The median concentration was calculated for each individual DNA. Individual DNA samples were then added to either case or control pools in equivalent molar amounts. Two separate ALS DNA pools, each consisting of 193 Caucasian ALS samples, were created in triplicate, generating a total of 6 independently created pools of ALS DNA samples. The control sub-pool of 700 Caucasians was also created de novo a total of three times to control for pipetting errors. Each of these ALS and control pools was hybridized to three Affymetrix 500K Mapping Arrays and two Illumina Infinium II HumanHap300 bead chip arrays following the manufacturers' protocols for genotyping individual DNA samples, yielding a total of 27 Affymetrix arrays and 18 Illumina arrays. There are a total of 766,955 unique SNPs between these two genotyping platforms with an average inter-marker distance of 3.9 kb, using both HapMap anchored tagSNPs on the Illumina platform and evenly distributed SNPs on the Affymetrix platform.

Significant ALS associated SNPs were identified as previously described using a silhouette statistic to rank differences in relative allele signals between cases and controls, implemented using GenePool Software (Melquistet al., Am J Hum Genet. 2007; 80:769-778; Pearson et al., Am J Hum Genet. 2007; 80:126-39). The top 192 ranked SNPs from the Affymetrix arrays and the top 192 ranked SNPs from the Illumina arrays were selected for validation in an independent validation group (384 SNPs total). Based on previous studies, this method is effective at identifying SNPs associated with disease, though a number of false positives are expected due to the added measurement variance from pooling, thus reinforcing the need for validation using individual genotyping on a separate case/control series. For each of the 384 validation SNPs, one additional SNP from the same Caucasian HapMap-defined haplotype block was chosen (r²>0.8) for genotyping in the validation group to ensure that we maximized the probability of having at least one informative SNP in the ethnically diverse replication series. Thus 2 SNPs per associated locus (768 SNPs in total) from the initial genome screen were tested in the validation population. Genotyping of these SNPs was contracted to k-biosciences.

For the 768 SNPs genotyped in the validation population, allelic χ² p-values were calculated using Haploview 3.32. SNPs failing Hardy-Weinberg equilibrium at a p=0.05 or that had a genotype call rate of lower than 0.05 were excluded from further analyses. The most significant association (rs6700125, p=1.8×10⁻⁵) passes the conservative Bonferroni corrected p-value cut-off of 1.3×10⁻⁴ (0.05/384 independent tests were performed since we consider 2 SNPs per haplotype block to be a single measurement point).

Immunoblot: _Frozen spinal cord tissue samples from autopsy confirmed 8 healthy non-neurologic controls and 10 ALS patients were obtained from the ALS Tissue Bank at the University of Pittsburgh. Equal ratio of males and females were used in each group. The average age for the control group was 60 years and the ALS group was 58 years. The average post-mortem interval was 8 hours for the control group and 6 hours for the ALS group. In addition, cerebrospinal fluid was obtained from healthy controls and ALS patients and frozen tissue samples from multiple organs of healthy control subjects were obtained following IRB consent from the University of Pittsburgh. Frozen tissue samples were homogenized in ice-cold lysis buffer using a polytron homogenizer and disposable plastic probes (Omni International, Marietta, Ga.). Lysis buffer contained 50 mM Tris (pH 8.0), 600 mM NaCl, 2% CHAPS and 1% protein inhibitor cocktail II (Sigma-Aldrich, St Louis, Mo.). Protein content of the homogenates was measured using BCA Protein Assay kit (Pierce, Rockford, Ill.). 50 μg of protein was loaded into each lane and electrophoresed on NuPage 12% Bis-Tris gels (Invitrogen, Carlsbad, Calif.) at 200 V. For cerebrospinal fluid (CSF) samples, 10 μl of CSF was loaded per lane (˜1 μg total protein per lane). Proteins were transferred onto nitrocellulose membranes (Bio-Rad, Hercules, Calif.), blocked in 5% non-fat milk, immunolabeled with mouse polyclonal antibody to FLJ10986 protein (Novus Biologicals, Littleton, Colo.) at 1:500 concentration in Tris buffered saline, pH 7.4 (TBS) containing 0.05% Tween 20, washed extensively in TBS, labeled with anti-rabbit HRP-labeled secondary antibody at 1:1,000 dilution in TBS, and after additional washes in TBS the antibody detected using Chemiluminescence reagent (PerkinElmer, Wellesley, Mass.).

### Example 2

### Whole Genome Association Screen

Genotyped SNPs from a screen of 386 ALS patients (155 female, 231 male; all Caucasian, mean age=59 years, mean ALSFRS-R=30.8) versus 700 control individuals (all Caucasian, mean age=68 years) were ranked by differences in pooled probe intensity data, as quantified by a silhouette statistic (Melquist et al. op. cit.; Pearson et al., op. cit.; Schymick et al., Lancet Neurol. 2007; 6:322-8). In previous studies, this statistic has been found to effectively identify SNPs with large reproducible allelic frequency imbalances in whole-genome associations on diseases with known association. Specifically, for both the Affymetrix and Illumina arrays, SNPs were ranked such that the marker with the highest silhouette statistic was assigned a ranking of 1 and the lowest scoring marker was assigned a ranking of 500,568 for the Affymetrix comparisons and 317,503 for the Illumina comparisons. The complete rank-ordered SNP list from both platforms is available at on the internet at the Tgen neurogeneitcs website (user name: reviewer, password: ALS) as Supplemental Tables 2 and 3. The 192 highest ranked SNPs from both Illumina and Affymetrix screens, plus an additional set of 384 adjacent tag SNPs on the identical Caucasian HapMap-defined haplotype block, were selected for subsequent replication in a completely independent replication series 1 population of 766 ALS patients (308 female, 458 male; 438 Caucasian, 136 minority, 192 unknown race; mean age=59.18; mean ALSFRS-R=29.94) and 750 neurologically normal controls (353 female, 397 male; all Caucasian; mean age=66.1).

### Example 2

### Validation of Significant Associations

Individual genotype data was obtained for 768 SNPs in the independent replication series 1 described above. Results showed significant association of 99/768 SNPs at the p<0.05 level, representing 76 unique loci (see FIG. 3, which presents Table 1). As expected, numerous loci were significant with both tag SNPs ensuring that genotyping error did not lead to any false associations as can be seen in Table 1. Of the 99 significant SNPs (p<0.05), 41 are intragenic or within ˜50 kb upstream or downstream of known or annotated genes (recognizing that linkage disequilibrium rates vary dramatically across different regions of the genome from a few kilo-bases to mega base-pairs, and that our ability to detect these distant associations is directly related to the strength of the association, the LD, and the informativeness of the marker). The remaining 35 loci tagged by these SNPs have no clear association with a known gene within these physical distance thresholds, although it is recognized that un-annotated regulatory elements and transcripts reside throughout the genome. Interestingly, 12 of the 41 annotated genes have functions related to cytoskeletal regulation or neuronal cell adhesion, suggesting that differences in these processes may play a critical role in the predisposition to sALS (see FIG. 3, Table 1).

Another completely independent sALS case and control series was used to validate the most significant 99 SNPs that had p<0.05 from replication series 1. Replication series 2 is a publicly available sALS genotyping data set performed on the Illumina Infinium II 550K genotyping platform (Schaper et al., Neurobiol Aging 2007, Mar. 10 Epub ahead of print). This data set was generated using samples available from the Coriell Cell Repository. 135 sALS patients were analyzed that were unique to their sample set (Coriell samples in common to both data sets were removed) and their 275 unique US-based Caucasian, non-Hispanic controls to determine if the loci that were identified were also associated with sALS in this additional independent data set. Because both the Illumina and Affymetrix platforms were used for the whole-genome genotyping, many of the top SNPs were not found in this third data set generated only on the Illumina platform. Thus, a locus-specific validation method was used where the Illumina Infinium II 550K SNPs present in each of the top significant loci (excluding the X chromosome) was identified using a window of 25 kilo base-pairs flanking the associated SNP and calculated the p values for all SNPs in the window in the data set generated by Schymick et al. (Lancet Neurol. 2007; 6:322-8). The most significant p-value at each locus, the replication series 2 SNP generating that p-value, and the chromosome position of that SNP within the respective locus was reported (FIG. 3, Table 1, last three columns). Results support the association of multiple loci with ALS in this additional independent data set, all of which are shown in FIG. 3, Table 1.

Complete odds ratio (OR) calculations for the statistically significant SNPs from replication series 1 (see FIG. 4, Table 1). Calculations were made using the DeFinetti program (available on the internet) and methods were adapted from Sasieni (Biometrics. 1997; 53:1253-1261). Tests including Hardy-Weinberg equilibrium tests for cases and controls, ORs calculated using the allele frequency difference between cases and controls, for heterozygotes, for homozygotes, allele positivity ORs, and the common ORs calculated using Armitage's trend test, along with statistical tests for significance of each are included. ORs referenced for specific associations below are those with the greatest magnitude, and the model associated with that OR. The Allele count ORs for genes which were statistically significantly associated with sALS across both replication series at a p<0.05 were FLJ10986 (rs6700125, OR=1.38, CI=1.16-1.65), PTPRT (rs13036957, OR=1.28, CI=1.04-1.56), ALK (rs4622670, OR=1.24, CI=1.04-1.47), PARP8 (rs27628, OR=1.20, CI=1.00-1.44) IL 18RAP (rs3771150, OR=1.27, CI=1.05-1.53), DSC3 (rs1314625, OR=1.22, CI-1.01-1.48), DGKB (rs38271, OR=1.18, CI=1.00-1.39), MAGI2 (rs757863, OR=1.24, CI=1.05-1.46), and LOXHD1 (rs988213, OR=1.31, CI=1.10-1.55). An additional 8 chromosomal loci were significant at the p<0.05 level in both replication series, but do not have gene annotations associated with them yet: 12q12 (rs1027615, OR=1.25, CI=1.04-1.50), 2q33.1 (rs12473579, OR=1.29, CI=1.09-1.53), 2q12.1 (rs17027230, OR=1.28, CI=1.06-1.53), 22q11.21 (rs4819840, OR=1.28, CI-1.07-1.54), 6q21 (rs7772593, OR=1.42, CI=1.05-1.93), 21q22.13 (rs2836061, OR=1.41, CI=1.13-1.77), 11p14.3 (rs7937375, OR=1.22, CI=1.04-1.44), 8p23.1 (rs7818421, OR=1.46, 0.90-2.36). All of these genes have statistically significant allelic imbalances between cases and controls across a total of three independent cohorts, yet the magnitude of effect is subtle. Thus, a multigenic additive model may account for the disease.

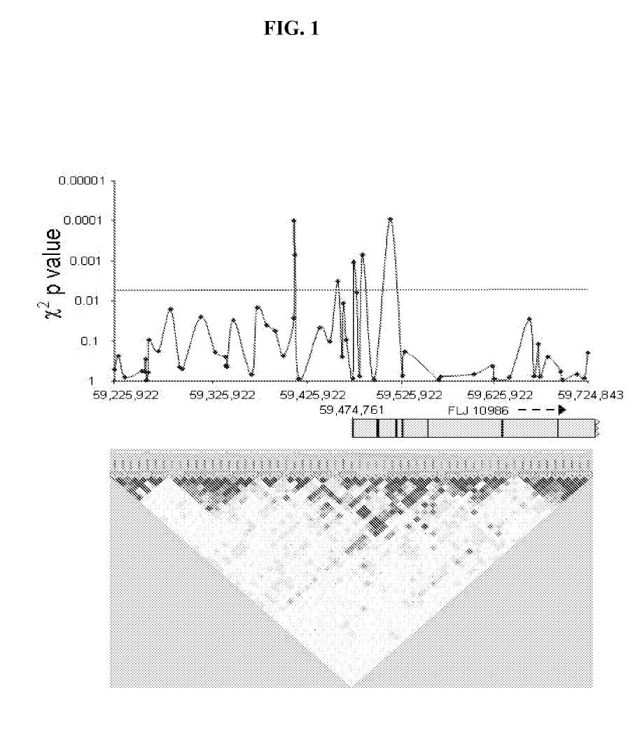

The most statistically significant associations in the analyses were with rs6700125 (p=0.000018) and rs6690993 (p=0.0002), which lie ˜60 kb upstream of an uncharacterized open reading frame named FLJ10986 (Table I). rs6700125 exceeds the conservative Bonferroni correction for the 384 independent tests (the second SNP per Haplotype block is not an independent measure and was thus not used to modify the correction threshold) performed on the replication group. Further, additional SNPs within this locus (rs7531917, p=0.04321 and rs6587852, p=0.04797) were associated with sALS in the replication series 2, a completely independently derived and generated data set of 135 sALS cases and 275 controls⁸ (see also FIG. 3, Table 1). To determine if the ALS association includes this novel gene, 71 additional flanking SNPs were genotype. These were derived by placing at least one informative SNP on each Caucasian HapMap-defined block spanning a total of 500 Kb across the locus (FIG. 1). Results showed four additional SNPs of high statistical significance (rs10493256, p=0.0033; rs6587852, p=0.0011; rs1470407, p=0.0007; rs333662, p=0.0000895), which lie in the promoter region and first two exons/introns of the FLJ10986 gene. These results show that the FLJ10986 gene is contained within the ALS associated region in the study population.

### Example 3

### Expression of FLJ10986 Protein

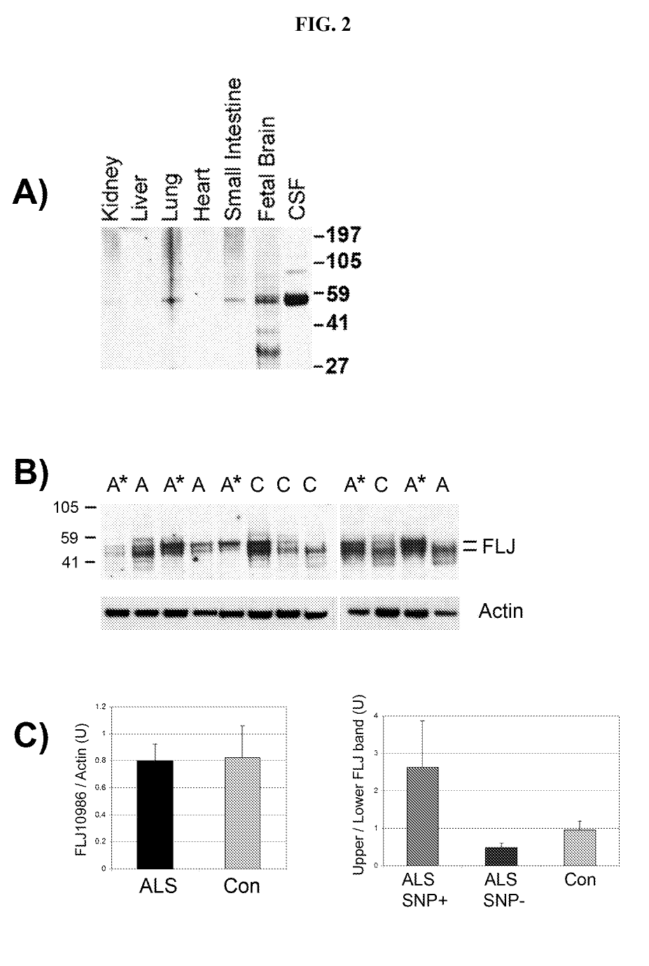

Since the most statistically significant genetic association was to an uncharacterized gene, studies were initiated to characterize the putative FLJ10986 gene product. A commercially available antibody to recombinant FLJ10986 protein was used for immunoblot analysis of various human organ tissues and cerebrospinal fluid to evaluate protein expression and also from spinal cord homogenates from control and ALS subjects. The predicted molecular mass of the FLJ10986 gene product is 48 kDa. A protein of approximately 48 kDa was observed in the kidney, lung and small intestine, with lower protein levels in the liver but absent from heart from healthy control subjects (FIG. 2). A protein doublet of approximately 48 and 50 kDa was evident in human fetal brain along with additional lower molecular weight species. Intense FLJ10986 immunoreactivity was also apparent in cerebrospinal fluid (FIG. 2A). As a control a gel with secondary antibody alone was labeled; these bands were not detected. A FLJ10986 protein doublet of approximately 45 and 48 kDa was evident in the spinal cord of control and ALS subjects (FIG. 2B). To confirm that these bands contain FLJ10986 protein, protein was immunoprecipitated from CSF using the anti-FLJ10986 antibody and analyzed the immunoprecipitate on a SDS-gel. The 45 kDa and 48 kDa bands were excised from the gel, proteins eluted and digested with trypsin. The tryptic fragments were sequenced using an Applied Biosystems, Inc. 4700 mass spectrometer (ABI 4700) via matrix assisted laser-desorption time-of-flight mass spectrometry (MALDI-MS-MS). Each of these bands contained FLJ10986 amino acid sequences, confirming that these gel bands correspond to FLJ10986 protein. While the total amount of FLJ10986 protein was equal in control and ALS spinal cord when normalized to the level of actin present in each sample, the relative ratio of the higher to lower molecular weight FLJ10986 immunoreactive bands was increased in ALS subjects that harbor either the rs6700125 or rs6690993 FLJ10986 polymorphisms (FIG. 2C). The FLJ10986 genotype of the control subjects was not available and therefore the presence or absence of FLJ10986 polymorphisms could not be ascertained in the control subjects. However the control subjects exhibit a ratio of upper to lower FLJ10986 protein bands more similar to the ALS patients lacking the FLJ10986 risk genotypes (FIG. 2C). These data indicate that the FLJ10986 protein is expressed in multiple human tissues, including cerebrospinal fluid.

### Example 4

### Subgroups of SNPs are Associated with Clinical Sub-Classifications of sALS

It is known that sporadic ALS (sALS) is clinically heterogeneous, and thus likely that there is molecular heterogeneity underpinning different sALS clinical subclasses. Because of this, it is also likely that overall SNP p-values may be diluted when assessing significance across a genetically and clinically diverse sALS series. Specific association analyses were performed based on multiple clinically relevant sub-classifications of sporadic ALS, since detailed clinical information was collected for the ALS patients enrolled in this study. These included analyses of male ALS patients versus female ALS patients, patients with bulbar onset ALS versus patients with limb onset ALS, and patients with early onset ALS (≦45 years) versus patients with late onset ALS (≦60 years). For each of these comparisons, specific subsets of the 768 SNPs used on the validation population showed highly statistically significant associations (FIG. 4, which presents Table 2). Comparison of the relevant clinical subclassifications to the control validation series allowed further clarification of the SNP associations. For example, rs735888 showed significant differences between female ALS patients and male ALS patients (p=0.0068). Separate comparisons of female ALS versus control and male ALS vs control were performed to determine if the SNP is specifically associated with ALS in either males or females (FIG. 4, Table 2, group B). rs735888 was associated with ALS in females (p=0.0246) and not with ALS in males (p=0.2808). Another finding was that some of the overall associations that observed in the complete validation population (FIG. 3, Table 1) were significant in certain subgroup comparisons and not others, suggesting that clinical subsets of ALS were driving the overall associations for these SNPs. For example, PARP8 had p-values of 0.026 and 0.0492 for the two SNPs genotyped (rs27628 and rs27248; Table 1). The p-value for these SNPs remained highly significantly associated with ALS in bulbar onset ALS (p=0.0036 and 0.0061, respectively) but were not associated with limb onset ALS (p=0.955 and 0.9373, respectively), indicating that these SNPs and the underlying PARP8 gene may be involved in site-of-onset differences in sALS. Similarly, rs155653676 (TIAM2) was significantly associated with early onset (<45 years) ALS (p=0.0044); but not with late onset (>60 years) ALS (p=0.3327). There are numerous similar findings for each comparison, all of which are summarized in FIG. 4, Table 2. This is the first identification of genetic associations with clinical sub-types in ALS.

It will be apparent that the precise details of the methods or compositions described may be varied or modified without departing from the spirit of the described invention. We claim all such modifications and variations that fall within the scope and spirit of the claims below.

1. A method for detecting a genetic predisposition to amyotrophic lateral sclerosis in a human subject, comprising
detecting the presence of a haplotype block comprising a tag single nucleotide polymorphism (SNP), wherein the tag SNP is
rs6690993, wherein position 59416003 is a G;
rs6700125, wherein position 59414818 is a T;
rs7074175, wherein position 20556984 is a T;
rs4827700 wherein position 145052081 is a G;
rs6036180 wherein position 22627977 is an A;
rs2836061 wherein position 38247104 is a C;
rs2279605 wherein position 55611622 is an A;
rs4756063 wherein position 33822142 is a G;
rs11018623 wherein position 88837360 is a G;
rs4629724 wherein position 121250591 is a T;
rs4704336 wherein position 75899375 is a G;
rs5970919 wherein position 22639221 is an A;
rs5929816 wherein position 136099981 is an A;
rs2279607 wherein position 55611764 is a T;
rs7003876 wherein position 1135748 is a T;
rs988213 wherein position 42378965 is an A;
rs2036535 wherein position 28775126 is a T;
rs5925683 wherein position 22629374 is a C;
rs10499100 wherein position 121250044 is a T;
rs1172149 wherein position 201956415 is a T;
rs3810715 wherein position 150555188 is a G;
rs13036957 wherein position 41255110 is a G;
rs752257 wherein position 22630289 is a G;
rs17027230 wherein position 102537848 is a C;
rs757863 wherein position 77316032 is an A;
rs10740320 wherein position 70840449 is a G;
rs4263905 wherein position 145052983 is a T;
rs10942784 wherein position 75889806 is an A;
rs10809959 wherein position 13497924 is a C;
rs10762294 wherein position 70840387 is a C;
rs1466471 wherein position 61478245 is a G;
rs3744477 wherein position 40183199 is a T;
rs10748358 wherein position 42149850 is a T;
rs12119273 wherein position 61655314 is a G;
rs10834819 wherein position 25821137 is a G;
rs10506228 wherein position 42150219 is a T;
rs12995017 wherein position 205046522 is an A;
rs945699 wherein position 224400054 is a G;
rs1554914 wherein position 150549225 is a T;
rs4287603 wherein position 2722492 is a G;
rs1027615 wherein position 41998556 is an A;
rs666481 wherein position 10010682 is a C;
rs1447830 wherein position 74695861 is a C;
rs12473579 wherein position 203030073 is a G;
rs905080 wherein position 41995195 is a G;
rs2205545 wherein position 150677351 is an A;
rs3771150 wherein position 102519369 is a C;
rs1891592 wherein position 148367576 is an A;
rs3749870 wherein position 155646464 is a G;
rs12279181 wherein position 25819399 is an A;
rs11172457 wherein position 56752884 is a G;
rs733281 wherein position 41264461 is a T;
rs4819840 wherein position 18096320 is an A;
rs4491817 wherein position 18097369 is a G;
rs 1314625 wherein position 26844530 is a C;
rs4516412 wherein position 203029371 is a G;
rs879012 wherein position 957788 is a G;
rs27628 wherein position 50266128 is a T;
rs276915 wherein position 26853979 is an A;
rs38271 wherein position 14080271 is a C;
rs276916 wherein position 26854159 is a C;
rs7772593 wherein position 106451750 is a T;
rs7937375 wherein position 21698795 is an A;
rs27248 wherein position 50268304 is an A;
rs4622670 wherein position 29357853 is a G; or
rs7818421 wherein position 8328291 is a C;
wherein the presence of the haplotype block determines the genetic predisposition to amyotrophic lateral sclerosis in the human subject.

2. The method of claim 1, wherein detecting the presence of the haplotype block comprises detecting a single nucleotide polymorphism with an r² value of 0.8 or greater from
rs6690993, wherein position 59416003 is a G;
rs6700125, wherein position 59414818 is a T;
rs7074175, wherein position 20556984 is a T;
rs4827700 wherein position 145052081 is a G;
rs6036180 wherein position 22627977 is an A;
rs2836061 wherein position 38247104 is a C;
rs2279605 wherein position 55611622 is an A;
rs4756063 wherein position 33822142 is a G;
rs11018623 wherein position 88837360 is a G;
rs4629724 wherein position 121250591 is a T;
rs4704336 wherein position 75899375 is a G;
rs5970919 wherein position 22639221 is an A;
rs5929816 wherein position 136099981 is an A;
rs2279607 wherein position 55611764 is a T;
rs7003876 wherein position 1135748 is a T;
rs988213 wherein position 42378965 is an A;
rs2036535 wherein position 28775126 is a T;
rs5925683 wherein position 22629374 is a C;
rs10499100 wherein position 121250044 is a T;
rs1172149 wherein position 201956415 is a T;
rs3810715 wherein position 150555188 is a G;
rs13036957 wherein position 41255110 is a G;
rs752257 wherein position 22630289 is a G;
rs17027230 wherein position 102537848 is a C;
rs757863 wherein position 77316032 is an A;
rs10740320 wherein position 70840449 is a G;
rs4263905 wherein position 145052983 is a T;
rs10942784 wherein position 75889806 is an A;
rs10809959 wherein position 13497924 is a C;
rs10762294 wherein position 70840387 is a C;
rs1466471 wherein position 61478245 is a G;
rs3744477 wherein position 40183199 is a T;
rs10748358 wherein position 42149850 is a T;
rs12119273 wherein position 61655314 is a G;
rs10834819 wherein position 25821137 is a G;
rs10506228 wherein position 42150219 is a T;
rs12995017 wherein position 205046522 is an A;
rs945699 wherein position 224400054 is a G;
rs1554914 wherein position 150549225 is a T;
rs4287603 wherein position 2722492 is a G;
rs1027615 wherein position 41998556 is an A;
rs666481 wherein position 10010682 is a C;
rs 1447830 wherein position 74695861 is a C;
rs12473579 wherein position 203030073 is a G;
rs905080 wherein position 41995195 is a G;
rs2205545 wherein position 150677351 is an A;
rs3771150 wherein position 102519369 is a C;
rs1891592 wherein position 148367576 is an A;
rs3749870 wherein position 155646464 is a G;
rs12279181 wherein position 25819399 is an A;
rs11172457 wherein position 56752884 is a G;
rs733281 wherein position 41264461 is a T;
rs4819840 wherein position 18096320 is an A;
rs4491817 wherein position 18097369 is a G;
rs1314625 wherein position 26844530 is a C;
rs4516412 wherein position 203029371 is a G;
rs879012 wherein position 957788 is a G;
rs27628 wherein position 50266128 is a T;
rs276915 wherein position 26853979 is an A;
rs38271 wherein position 14080271 is a C;
rs276916 wherein position 26854159 is a C;
rs7772593 wherein position 106451750 is a T;
rs7937375 wherein position 21698795 is an A;
rs27248 wherein position 50268304 is an A;
rs4622670 wherein position 29357853 is a G; or
rs7818421 wherein position 8328291 is a C.

3. The method of claim 1, wherein determining the genetic predisposition to amyotrophic lateral sclerosis is detecting amyotrophic lateral sclerosis in the subject.

4. The method of claim 1, wherein detecting the genetic predisposition to amyotrophic lateral sclerosis is determining the risk of developing amyotrophic lateral sclerosis in a subject who does not currently have symptoms of amyotrophic lateral sclerosis.

5. The method of claim 1, wherein the method comprises detecting the presence of at least five different haplotype blocks each comprising a different one of the tag SNPs.

6. The method of claim 1, wherein the method comprises detecting the presence of at least ten different haplotype blocks each comprising a different one of the tag SNPs.

7. The method of claim 1, wherein the method comprises detecting the presence of at least twenty different haplotype blocks each comprising a different one of the tag SNPs.

8. The method of any one of claims 1-7, wherein detecting the presence of the haplotype block comprises detecting the presence of one or more of the tag SNPs that identifies the haplotype block.

9. The method of claim 8, wherein the method comprises detecting the presence of at least five of the tag SNPs.

10. The method of claim 8, wherein the method comprises detecting the presence of at least ten of the tag SNPs.

11. The method of claim 1, wherein the method comprises detecting the presence of at least twenty of the tag SNPs.

12. The method of claim 1, wherein the haplotype block comprises a nucleic acid encoding FLJ10986.

13. The method of claim 1, comprising detecting all of the tag SNPs.

14. A method of detecting the genetic predisposition of a subject to bulbar onset amyotrophic lateral sclerosis, comprising, detecting the presence of a haplotype block comprising a tag single nucleotide polymorphism (SNP), wherein the tag SNP is
rs12695988 wherein position 154604997 is an A;
rs4680060 wherein position 154601610 is a T;
rs988213 wherein position 42378964 is a G;
rs10884751 wherein position 111100812 is an A;
rs7806370 wherein position 38461063 is a C;
rs6677714 wherein position 236530180 is an A;
rs2247691 wherein position 41199732 is T;
rs11233487 wherein position 82529791 is a T;
rs 17667053 wherein position 70704931 is a C;
rs7193888 wherein position 82653630 is a T;
rs27628 wherein position 50266127 is a T;
rs27248 wherein position 50268303 is an A;
rs17741655 wherein position 127147541 is a G;
rs4745434 wherein position 75515725 is a T;
rs13398914 wherein position 127152871 is an A;
rs7740727 wherein position 5654334 is a G;
rs11711863 wherein position 185808656 is a C; or
rs3944131 wherein position 92386146 is a C;
wherein the presence of one or more of the haplotype blocks determines the genetic predisposition to bulbar onset amyotrophic lateral sclerosis in the human subject.

15. The method of claim 14, wherein detecting the presence of the haplotype block comprises detecting a single nucleotide polymorphism with an r² value of 0.8 or greater from
rs12695988 wherein position 154604997 is an A;
rs4680060 wherein position 154601610 is a T;
rs988213 wherein position 42378964 is a G;
rs10884751 wherein position 111100812 is an A;
rs7806370 wherein position 38461063 is a C;
rs6677714 wherein position 236530180 is an A;
rs2247691 wherein position 41199732 is a T;
rs11233487 wherein position 82529791 is a T;
rs17667053 wherein position 70704931 is a C;
rs7193888 wherein position 82653630 is a T;
rs27628 wherein position 50266127 is a T;
rs27248 wherein position 50268303 is an A;
rs17741655 wherein position 127147541 is a G;
rs4745434 wherein position 75515725 is a T;
rs13398914 wherein position 127152871 is an A;
rs7740727 wherein position 5654334 is a G;
rs11711863 wherein position 185808656 is a C; or
rs3944131 wherein position 92386146 is a C.

16. The method of claim 14, wherein determining the genetic predisposition to bulbar onset amyotrophic lateral sclerosis is detecting bulbar onset amyotrophic lateral sclerosis in the subject.

17. The method of claim 14, wherein determining the genetic predisposition to bulbar onset amyotrophic lateral sclerosis is determining the risk of developing bulbar onset amyotrophic lateral sclerosis in a subject.

18. The method of claim 14, wherein the method comprises detecting the presence of at least five different haplotype blocks each comprising a different one of the tag SNPs.

19. The method of claim 14, wherein the method comprises detecting the presence of at least ten different haplotype blocks each comprising a different one of the tag SNPs.

20. The method of claim 14, wherein the method comprises detecting the presence of at least twenty different haplotype blocks each comprising a different one of the tag SNPs.

21. The method of any one of claims 14-19, wherein detecting the presence of the haplotype block comprises detecting the presence of one or more of the tag SNPs that identifies the haplotype block.

22. The method of claim 21, wherein the method comprises detecting the presence of at least five of the tag SNPs.

23. The method of claim 21, wherein the method comprises detecting the presence of at least ten of the tag SNPs.

24. The method of claim 22, wherein the method comprises detecting the presence of at least twenty of the tag SNPs.

25. The method of claim 21, comprising detecting all of the tag SNPs.

26. A method of determining if a subject has a genetic predisposition to early onset amyotrophic lateral sclerosis, comprising, detecting the presence of a haplotype block comprising a tag single nucleotide polymorphism (SNP), wherein the tag SNP is
rs12471471 wherein position 213848557 is an A;
rs7569588 wherein position 45331732 is a T;
rs12929266 wherein position 49453731 is a T;
rs1390762 wherein position 49452674 is an A;
rs11096490 wherein position 17949476 is a G;
rs4245528 wherein position 106480927 is an A;
rs 17118549 wherein position 59196347 is a T;
is 16983965 wherein position 17951571 is a T;
rs10438441 wherein position 90663620 is a T;
rs2919708 wherein position 70660625 is a G;
rs11089823 wherein position 35833678 is a C;
rs38271 wherein position 14080270 is a C;
rs838732 wherein position 234103751 is a T;
rs2010435 wherein position 82528143 is an A;
rs11233487 wherein position 82529791 is an A;
rs7171883 wherein position 90664487 is an A;
rs2093689 wherein position 94150134 is an A;
rs11914132 wherein position 35833586 is a T;
rs9558712 wherein position 105646374 is a G;
rs3020040 wherein position 70661830 is an A;
rs838731 wherein position 234097362 is a C;
rs11751085 wherein position 155653676 is a C;
rs10224956 wherein position 32969593 is a G;
rs3936139 wherein position 2538575 is a C;
rs7467398 wherein position 7392207 is a G;
rs6772591 wherein position 171997451 is a G;
rs13236414 wherein position 32969673 is a G; or
rs1943934 wherein position 69938052 is an A;
wherein the presence of one or more of the haplotype blocks determines the genetic predisposition to early onset amyotrophic lateral sclerosis in the human subject.

27. The method of claim 14, wherein detecting the presence of the haplotype block comprises detecting a single nucleotide polymorphism with an r² value of 0.8 or greater from
rs12471471 wherein position 213848557 is an A;
rs7569588 wherein position 45331732 is a T;
rs12929266 wherein position 49453731 is a T;
rs1390762 wherein position 49452674 is an A;
rs11096490 wherein position 17949476 is a G;
rs4245528 wherein position 106480927 is an A;
rs17118549 wherein position 59196347 is a T;
rs16983965 wherein position 17951571 is a T;
rs10438441 wherein position 90663620 is a T;
rs2919708 wherein position 70660625 is a G;
rs11089823 wherein position 35833678 is a C;
rs38271 wherein position 14080270 is a C;
rs838732 wherein position 234103751 is a T;
rs2010435 wherein position 82528143 is an A;
rs11233487 wherein position 82529791 is an A;
rs7171883 wherein position 90664487 is an A;
rs2093689 wherein position 94150134 is an A;
rs11914132 wherein position 35833586 is a T;
rs9558712 wherein position 105646374 is a G;
rs3020040 wherein position 70661830 is an A;
rs838731 wherein position 234097362 is a C;
rs11751085 wherein position 155653676 is a C;
rs10224956 wherein position 32969593 is a G;
rs3936139 wherein position 2538575 is a C;
rs7467398 wherein position 7392207 is a G;
rs6772591 wherein position 171997451 is a G;
rs13236414 wherein position 32969673 is a G; or
rs1943934 wherein position 69938052 is an A.

28. The method of claim 27, wherein determining the genetic predisposition to amyotrophic lateral sclerosis is detecting early onset amyotrophic lateral sclerosis in the subject.

29. The method of claim 27, wherein determining the genetic predisposition to early onset amyotrophic lateral sclerosis is determining the risk of developing early onset amyotrophic lateral sclerosis in a subject.

30. The method of claim 27, wherein the method comprises detecting the presence of at least five different haplotype blocks each comprising a different one of the tag SNPs.

31. The method of claim 27, wherein the method comprises detecting the presence of at least ten different haplotype blocks each comprising a different one of the tag SNPs.

32. The method of claim 27, wherein the method comprises detecting the presence of at least twenty different haplotype blocks comprising a different one of the tag SNPs.

33. The method of any one of claims 27-33, wherein detecting the presence of the haplotype block comprises detecting the tag SNP.

34. The method of claim 33, wherein the method comprises detecting the presence of at least five of the tag SNPs.

35. The method of claim 33, wherein the method comprises detecting the presence of at least ten of the tag SNPs.

36. The method of claim 33, wherein the method comprises detecting the presence of at least twenty of the tag SNPs.

37. The method of claim 33, comprising detecting all of the tag SNPs.
